(19) United States
(12) Patent Application Publication
(10) Pub. No.: US 2021/0018489 A1
(43) Pub. Date: Jan. 21, 2021

(54) Apparatus and Method for Detecting and Correcting Blood Clot Events

(71) Applicants: Jahan Razavi, Saratoga, CA (US); Mohammad Amin Arbabian, San Francisco, CA (US)

(72) Inventors: Jahan Razavi, Saratoga, CA (US); Mohammad Amin Arbabian, San Francisco, CA (US)

(73) Assignees: Jahan Razavi, Saratoga, CA (US); Mohammad Amin Arbabian, San Francisco, CA (US)

(21) Appl. No.: 16/513,306

(22) Filed: Jul. 16, 2019

(51) Int. Cl. G01N 33/49; G01N 33/86; C12Q 1/56

(52) U.S. Cl. CPC G01N 33/4905; G01N 33/582; C12Q 1/56; G01N 33/86

(57) ABSTRACT

An apparatus to detect blood clots based on the analysis of the blood's chromatic properties is described. The chromatic property can be determined non-evasively when used in conjunction with ECMO systems. The red, green, and blue chromatic values of a clotting site and a reference site are compared to determine if a clotting event occurred. It was discovered that, at a minimum, only the red chromatic value needs to be tested and measured to determine if a clotting event had occurred. This system can be adopted to monitor clot formation in heart surgery, heart or lung transplants or patients coupled to ECMO requiring an ability to measure the clots to a blood depth of 20 mm. Once clots are detected, the system can introduce anti-coagulants into the blood stream to reduce the clot formation.

### CROSS-REFERENCE TO RELATED APPLICATIONS

N/A.

### BACKGROUND OF THE INVENTION

Our body's blood system is very resilient. If the blood vessels become wounded or damaged, coagulation or clotting of the blood starts to occur almost immediately to help repair the system. There are many causes of injuries that include a fall, car crash, a sporting accident, or the surgical cuts due to a scheduled or unscheduled medical procedure, etc. For the latter case, blood clot formation, also called blood coagulation, can occur during some of these medical procedures. The injury process activates the blood system to increase platelets activity and start fibrin formation. These components are required in the clotting process. The platelets group together and attempt to plug up any injured site, while the fibrin strands are generated which adhere to the plug reinforcing the initial plug and adding strength to the plug. The blood system being very resilient attempts to repair itself. The clots attempt to seal off any openings in the blood vessels. The clot components also help to gel up the blood and transform the liquid blood into solid plugs. These plugs attempt to seal up any tears in the blood vessel. The clotting process reduces bleeding in the patient who has experienced an ‘open’ wound and is one of the positive lifesaving attributes of clotting.

During surgery, the clotting process may never achieve its goal of stopping the patient from bleeding. The body's natural response is to create clotting to repair any wounds. In some cases, the formation of these clots can cause the patient to swell and, in other cases, if clot formation is not controlled, death can occur. An out of control clotting process is a negative attribute of clotting. The clotting process must always be under control to insure the safety of the patient.

In certain medical procedures such as transplants of organs, heart surgery, or lung surgery, dialysis, hemofiltration, extracorporeal (occurring outside the body) blood circulation techniques are required. Extracorporeal blood circulation is a procedure that extends the life of a patient by actively removing blood from a patient, processing that blood by adding oxygen through a membrane, removing carbon dioxide and heating the blood before infusing the blood back into the patient. Extracorporeal membrane oxygenation (ECMO) is one type of an extracorporeal blood circulation system. The blood's flow rate is around 2 liters per minute in an ECMO system. Another version of ECMO also includes a pump that can be used to by-pass or aid the pumping ability of the patient's heart. ECMO offers two types of live saving techniques: veno-venous and veno-arterial.

Veno-venous is any technique in which blood is taken from a vein, the extracted blood is processed, and then the blood is returned back to the patient via a vein. For example, the right internal jugular vein receives blood from the lung while the femoral vein in the upper leg provides blood to the lung. The veins at these two locations can be cut surgically. Next, plastic tubes about 20 mm in diameter called cannulas are inserted into the openings of the surgically cut veins on the side of the neck and at the top of the leg allow an easy access to the patient's blood supply system. The blood flows away from the patient via the femoral vein, the blood moves through the coupled cannulas to the artificial lung, the blood moves through a warmer to heat the blood, and then the blood finally moves into to the patient's jugular vein which then routes the oxygenated blood into the body. Now, the blood by-passes the lung of the patient and instead is oxygenated by the external artificial lung siting besides the body. The artificial lung (oxygenator, RGB Detector, Heat Exchange, etc.) emulates the lung's functions by removing carbon dioxide and adding oxygen to the blood as it is flowing through the system. Operative procedures can now be performed on the lung: surgery; transplant, or some form of special treatment or preparation. Veno-venous is critical for patients whose lungs have little ability to pump or create oxygen.

Veno-arterial takes blood from a vein, the extracted blood is processed, then the blood is returned back to the patient via an artery. This type of ECMO provides support for both the lungs and the heart. Since this setup bridges the patient's heart (receives blood from a vein and delivers to an artery), a ‘pump’ is added to the external circulation path to help pump some or all of the blood and thereby aids the heart to perform its ‘pumping’ function. In one case, two cannulas are placed in large vessels on the side of the neck. The tips are positioned such that the tip of one cannula is in the femoral artery while the other tip is near the inferior vena cava. The ECMO machine takes blood from the vein, adds oxygen and removes carbon dioxide, warms the blood and then returns the blood to the artery. The system also “pumps” the blood through the body reducing or by-passing the need for the heart to perform this pumping. Operative procedures can now be performed on the heart and/or lungs: surgery; transplant, or some form of special treatment or preparation. Veno-arterial is critical for patients whose heart is falling to pump and/or whose lungs have little ability to pump or create oxygen.

During the surgery, the body, in response, generates blood clots. The formation of these blood clots can be a serious issue if not contained. Some of the formed clots can adhere to the sides of the blood vessel's wall. This constricts the flow of blood causing more blood clots to pass through the narrower openings. The passing clot adheres to the walls and further reduces the size of the opening. Reducing or stopping the blood flow to any portion of the body is a situation that should be avoided.

Bodily injury (trauma, surgery) initiates the generation of blood clots within the circulatory system. Blood clots occur when the blood thickens and they coalesce and form a clump, mass, or clot. These clots can travel to other parts of the body. During medical procedures that involve extracorporeal blood circulation, such as: heart surgery, organ transplant, etc., the requirement to contain blood clot formation within allowable bounds must be met. An excessive formation of clots can cause the patient to swell and in other cases, if clot formation is not controlled, death can occur. Once the blood clot events exceed a specified blood clot level, preventive measures needs to be employed to prevent the patient from experiencing large blood clot levels during and after the procedure.

Other detection techniques to prevent blood clot formation exist. Previous clot detection techniques monitor the optical, ultrasound, or impedance properties of the blood. Unfortunately, these techniques face some shortcomings, in a first reference “Optical coherence tomography to investigate optical properties of blood during coagulation,” Journal of Biomedical Optics, vol. 16, pp. 1-7, September 2011 by X. Xu et al. use an optical coherence tomography technique to measure blood opacity. However, it encounters two drawbacks. OCT is relatively slow, with only a few frames per second, while the penetration depth is limited to a few millimeters. In a second reference, “A novel ultrasound-based method to evaluate hemostatic function of whole blood,” Clinica Chimica Acta, vol. 411, pp 106-113, October 2009 by F. Viola et al shows the use of ultrasound pulses to measure the viscoelasticity of blood. This method relies on transmitting pulses in a narrow beam to cause deformations in blood, which would lead to a narrow aperture and hence a need for a large number of ultrasound devices to spread across the entire width of the tube. In another reference, “Assessing blood coagulation status with laser speckle rheology,” Biomedical Optics Express, vol. 5, no. 3, February 2014, by M. M. Tripathi use laser speckle rheology to measure viscoelasticity. However, the penetration depth of laser is limited; as explained in “Laser Energy and Dye Fluorescence Transmission through Blood in Vitro,” American Journal of Ophthalmology, vol. 119, pp. 452-457, April 1995, by S. M. Cohen, shows that laser energy diminishes to 14% after 500 μm. This would mean that, after 2 mm of blood depth, the laser power drops by roughly 34 dB. Also, the laser spot size is around 100 μm. This would necessitate the use of many lasers or rapid scanning of the full width of the tube, making an actual system more expensive. These systems require a plurality of identical test devices to measure each portion of the full width of the tube. Many of these techniques fail to make a cost efficient system since the need to penetrate and analyze the full width of the thick samples of blood requires a plurality of test units each unit carrying a price tag.

An ability to easily and quickly determine blood clot events that occur within a circulatory system is desirable. Ideally, if measurements can be performed using a non-evasive technique, then the system becomes very portable. Ability to determine clotting events in thick samples of blood is necessary. The adjective ‘thick’ refers to a dimension. The system must use a simple and a reliable measuring apparatus to perform this task of clot detection in thick blood systems.

### BRIEF SUMMARY OF THE INVENTION

After initial experimentation, the hypothesis that the color composition of blood in terms of red, green, and blue (RGB) values can serve as an early indicator of coagulation proved to be correct. Blood clots were detected by analyzing the chromatic properties of the blood and comparing the results to a reference image.

In one embodiment, capturing the images of the blood samples aids in analysis of blood clots. The blood moves at around 2 liters per minute in typical extracorporeal circuits. There is not enough time to withdraw blood from the cannula and test it for clots. Instead, images of the blood sample will be captured and analyzed. Besides determining if blood clots occur, this collected and analyzed information can be shared with others in a common database that can be accessible by all. This database can be used in machine learning to further advance the understanding of blood clots.

In another embodiment, the reference image is generated by propagating light through a virgin blood sample from a patient. The virgin sample would be clot-free. The transmitted light is captured in a camera comprising red (R), green (G), and blue (B) sub-pixels. The three separate color images (RGB) of the virgin blood sample are labeled accordingly. These images can be stored into memory and are labeled the reference (RGB) images. Light is then propagated through a blood sample from that patient who is now experiencing blot clotting. The transmitted light is captured in the same camera comprising red (R), green (G), and blue (B) sub-pixels. These images can be stored into memory and are labeled the clotting (RGB-1, RGB-2 . . . ) images. Each of the three different colored pixel images were individually examined and compared to their corresponding reference image. It was discovered that the red (R) color image result predicts the presence of the blood clot events more effectively than when compared to the green (G) or blue (B) color images comparisons.

In another embodiment, the reference image is generated by propagating light through a virgin blood sample from a patient. The transmitted light is captured in a camera comprising red (R), green (G), and blue (B) sub-pixels but only the red (R) sub-pixel values are used as a reference. The red color image (R) of the virgin blood sample is labeled the reference (R) image. Light is then propagated through a blood sample from that patient who is now experiencing blot clotting. The red (R) transmitted light is stored into memory. A time sequence of (R) images can be made and labeled the clotting (R-1, R-2 . . . ) images. Each of the clotting images can be compared to the reference (R) image providing a time sequence of the clotting event. As blood is being clotted over time, the clotting can be measured directly by determining how much the transmissivity of the red (R) transmitted light decreases in comparison to the reference (R) image.

In another embodiment, the clotting event is captured in an image. The image is captured by an array of pixels arranged in rows and columns on a planar surface. Each sequence of images provides both temporal and spatial information. The spatial information tells where the clots are located, how many clots exist, what percentage of the area do the clots occupy, etc. The temporal information tells how the clot count changes over time, how the density of clots increases, how the density of clots decreases, how the clot's move, what is the clot's velocity, their acceleration, etc.

In another embodiment, several methods of improving the estimate of the clot size using the temporal and spatial information are described. The clotting event is captured in an array of pixels arranged in rows and columns on a planar surface. The pixels array sizes are offered in a variety of pixel x by y sizes. The Basler camera has a pixel array sized as 640 pixels×480 pixels and takes 750 images per second (every 1.3 ms). Other arrays exist which have x-y dimensions of 720×480, 1280×720, 1920×1080, etc., and different frame rates. A sequence of images is taken to provide both temporal and spatial information. The spatial information comprises the grid coordinates corresponding to the x-y position of the pixel containing the blood clot. The temporal information is contained within the sequence of the images. For example, in one embodiment, a blood clot has been identified in pixel (100, 200) in image 1. In image 2, the same blood clot has been identified in pixel (100, 400). In image 3, the same blood clot has been identified in pixel (100, 600). One analysis determines the velocity of the clot as 200 pixels/1.3 ms using the temporal and spatial information. Another analysis measures and compares the different images of the same clot in images 1-3, calculates the areas in each of the images 1-3, and averages the three values (n, in the general cas) of the blood clot areas to get an average blood clot size. Yet another analysis monitors the area of the clot over a sequence of images to determine if the clot size is increasing or decreasing using both the temporal and spatial information.

In another embodiment, it would be desirable to detect clots within a segment of blood. Blood clot detection in extracorporeal circuits presents several challenges. The first is that the inner diameter of the cannula (tube) used in extracorporeal blood circulation circuits such as ECMO is large, roughly 20 mm. Ideally, the light source should be bright enough to penetrate the thickest portion of the blood flow (the light passing along the diameter of the cannula) but there is a large loss in the magnitude of the light's intensity. Instead, a plurality of detectors is used to receive the light from different portions of the cannula after being transmitted through the blood. These detectors can be arranged around the circumference of the cannula, each detector detects light from a different portion of blood within the cannula. In one embodiment, the detector can be designed to capture all the light from cannula. In other embodiments, the detector can be designed to observe one or more sub-portions of the cylindrical section.

In another embodiment, the clot measurement apparatus is coupled non-evasively to an extracorporeal circulation system via the transparent cannula. The transmitted light through the cylindrical blood sample provides a first image. After each new time increment, another image is taken and stored as clot (R1, R2, R3 . . . ) images. These images can be compared to the reference (R) image to determine the clotting data: clot count, clot size, clot clusters, clot density, clot velocity, etc. The clotting data can be used to make decisions. For example, a clot density of 10% (or any other selected value) can be set as a threshold value to perform an action. If the clot density is always less than 10%, then the clot density is less than the threshold value, so do nothing. However, if the clot density increases above 10%, then inject an anti-coagulant into the patient (intravenously, for example) with the intent of decreasing the clot density below 10%. An anti-coagulant decreases the clotting of the blood and helps to prevent the formation of further clots. After waiting a time period, the injection fluid disperses throughout the patient, and the clot density can be measured again. If the clot density is less than 10%, then the clot density is less than the threshold value and the patient has returned safely within bounds. The system then continues monitoring the patient to insure the value of the clot density remains safely within bounds.

In another embodiment, the clot measurement apparatus is coupled non-evasively to an extracorporeal circulation system via the transparent cannula. The transmitted light through the cylindrical blood sample provides a first image. After each new time increment, another image is taken and stored as clot (R1, R2, R3 . . . ) images. These images can be compared to the reference (R) image to determine the clotting data: clot count, clot size, clot clusters, clot density, clot velocity, etc. The clotting data can be used to make decisions when dangerous levels of clots are detected. For example, a clot density of 80/can be set as an upper limit to indicate that the patient is approaching a critical situation (death). If the clot density reaches 80%, identify the criticalness of this situation to all involved performing this procedure by raising an alarm.

In another embodiment, the clot measurement apparatus is coupled non-evasively to an extracorporeal circulation system via the transparent cannula. The transmitted light through the cylindrical blood sample provides a first image. After each new time increment, another image is taken and stored as clot (R1, R2, R3 . . . ) images. These images can be compared to the reference (R) image to determine the clotting data: clot count, clot size, clot clusters, clot density, clot velocity, etc. The clotting data can be used to make decisions. For example, a clot size of 0.1 mm (or any other selected value) can be used as a threshold value to perform an action. If the clot size is always less than 0.1 mm, then the clot size is less than the threshold value, so do nothing. However, if the clot size increases above 0.1 mm, then inject an anti-coagulant into the patient (intravenously, for example) with the intent of decreasing the clot size below 0.1 mm. After waiting a time period, the injection fluid disperses throughout the patient, and the clot size can be measured again. The clot size is measured again. If the clot size is less than 0.1 mm, then the clot size is less than the threshold value, the patient is safely within bounds. The system then continues monitoring the patient to insure the value of the clot size remains safely within bounds.

In another embodiment, a plurality of sensors surrounding a blood sample is measuring the light response to one or more light sources illuminating the blood sample. The sensors can be positioned around the circumference of the cannula, along the length of the cannula, and/or along the length and circumference of the cannula. The light sources can be positioned around the circumference of the cannula, along the length of the cannula, and/or along the length and circumference of the cannula.

In another embodiment, the sensors can be positioned randomly/uniformly around the circumference of the cannula, along the length of the cannula, and/or along the length and circumference of the cannula. The light sources can be positioned randomly/uniformly around the circumference of the cannula, along the length of the cannula, and/or along the length and circumference of the cannula. Many other light source/sensor configurations are possible. Once the concept of the idea has been embraced, many additional embodiments of different configurations include: a single light source but multiple detectors to sense light transmitted through different portions of the cylindrical slab; or multiple light sources but only one sensor to pick up summed value of the multiple light sources. The possibilities can be easily extended further.

In another embodiment, at least one light source can be used to illuminate a cylindrical or rectangular volume of blood. Sensors (detectors) can be positioned around the circumference or perimeter of the tube, each sensor sensing the transmissivity, reflectivity or transmissivity and reflectivity of blood within a section of the volume of the blood to a depth of about 10 mm. The sensors outputs are compared with a reference value to determine if clotting is occurring. The sensors can detect clotting in tubes having a diameter or side dimensions of 20 mm.

In another embodiment, a portion, the entire RGB detector unit, or a plurality of RGB detectors are placed inside the cannula. In one example, the light source can be external to the cannula while the camera is embedded within the blood flow inside the cannula to collect data. In another example, the light source and camera are placed internally to the cannula. Both light source and camera may be powered and controlled wirelessly. Wireless signals can be applied to the unit to deliver power to the unit while other wireless signals can be used to communicate control/data information to orchestrate the system.

In another embodiment, supervised machine learning (ML) accompanied by numerous data can be used to train the RGB detector to recognize various clotting events, such as, clot occurrence, clot size, clot count, clot density, clot clusters, clot velocity, threshold levels, etc. Once trained, the ML can determine when a ‘threshold’ has been exceeded and then perform a corrective procedure. For example, if a clot cluster threshold of ‘4’ has been set, ML detects and identifies that the clot cluster exceeded ‘4’. In addition, ML has determined and implemented the corrective procedure by adding a particular amount of anti-coagulant. In addition, the clotting data can be stored into memory and used with machine learning to make predictions about the system.

In other embodiments, non-supervised ML can be used to train the RGB detector to recognize the various clotting cluster and grouping events, such as, clot occurrence, clot size, clot count, clot density, clot clusters, clot velocity, threshold levels, etc. The ML can determine when a ‘threshold’ has been exceeded and then perform a corrective procedure. For example, if a clot density threshold of ‘2’ has been set, ML detects and identifies that the clot density exceeded ‘2’. In addition, ML has determined and implemented the necessary corrective procedure.

In another embodiment, a special camera can be manufactured where the green and blue sub-pixels are substituted with red sub-pixels. Now all sub-pixels in the chip are red. The red sub-pixels are tripled in count. The camera offers an improvement in the accuracy of the camera and reduces silicon area waste.

In another embodiment, the RGB detector monitors, measures, and stores the transmissivity of blood for the primary red color (R) in real-time, early detection of blood clots in blood flows. The RGB detector can also be instructed to monitor, measure, and store the results for the green (G) and blue (B) colors, for later reference. Several embodiments are presented using this RGB detector to detect clotting events, to detect their concentration, to use feedback monitor coagulation, and use injection of anti-coagulant to control the clotting events in a biological specimen. The way to perform the last embodiment is monitor the blood flows for blood clots, once detected determine if a threshold is exceeded, if so, introduce an anti-coagulant into the blood system.

In both veno-venous and veno-arterial methods, the cannulas (transparent tubes) of the system are exposed in an extracorporeal blood circulation environment allowing visual access to the current blood of the patient. In some embodiments, an RGB Detector can be coupled to the cannula to obtaining information on the current clotting blood state of the patient. The exposed transparent tubes in an extracorporeal blood circulation environment allow for the possibility of analyzing the blood without invasive procedures. The RGB Detector can be placed around the cannula without disturbing the veno-venous and veno-arterial system's operating procedures.

In another embodiment, the shape of a cannula can be made with a rectangular cross section rather than the circular cross section such that the transmitted light from light source to the camera of the RGB detector would pass through equal volumes of blood.

In another embodiment, the light-detector system is a non-invasive technique for a patient using ECMO. Transparent cannulas are used in ECMO procedures and the light-detector system can be configured to view the blood flow within the cannula. This offers contactless detection, avoids disturbing the circulation system, and allows for higher portability.

In another embodiment, clots could be detected with a spatiotemporal matched filtering method, similar to algorithms utilized for array imaging systems, for example holographic techniques, phased arrays, angle of arrival estimation techniques, etc. This method resembles coherent combining of signals across an imaging aperture, taking into account the phase or time shift from the spatial distribution of sensing elements. For more complex flow (e.g. non-laminar) and/or with unknown velocity of particles, more advanced algorithms, including but not limited to iterative methods, joint estimation of particle location and velocity, and other hypothesis-driven techniques can be used to further enhance the detection capability.

In another embodiment, a system for a measurement of clot formation in blood of a patient comprising: at least one light source arrangement providing an electromagnetic radiation in a visible spectrum range, an infrared spectrum range, or both spectrum ranges; a light transmission arrangement for channeling the electromagnetic radiation through, or reflected from, the blood of the patient; one or more light detection arrangements receiving the channeled electromagnetic radiation from the light transmission arrangement to capture amplitudes over a frequency range of the channeled electromagnetic radiation; and a computation device arrangement computing spatial, temporal, or spatial and temporal analysis on the captured amplitudes corresponding to a pixel data output value of the light detection arrangement, wherein an anti-coagulant is injected into the blood of the patient when the pixel data output value exceeds a reference pixel data output value. The system wherein the light detection arrangement, further comprises: a detector comprised of a plurality of pixels arranged in rows and columns on a planar surface; a lens to focus the channeled electromagnetic radiation onto the plurality of pixels, the radiation incident substantially perpendicular to the planar surface, wherein each pixel is subdivided into a Red, a Green, and a Blue sub-pixel, and blood clotting can be detected by using the Red sub-pixel data of the pixel data output value from the light detection arrangement. The system wherein the computation device arrangement, further comprises: a memory to store a plurality of pixel data output values; a comparator to compare a reference pixel data output value and the channeled electromagnetic radiation of the specimen to detect a clotting event, wherein hardware to perform the comparator operation is selected from the group consisting of a field programmable gate array (FPGA), a multi-core central processing unit (MC-CPU), a graphics processing unit (GPU), and a machine learning (ML) device. The system wherein a plurality of light detection arrangements are distributed around the blood of the patient, each of the light detection arrangements detecting blood clots to a depth of at least of 10 mm. The system wherein the blood of the patient being measured flows within either a cannula of an extracorporeal blood circulation system, a vein of the patient, or an artery of the patient.

In another embodiment, a system for a measurement of clot formation in blood of a patient comprising: at least one light source arrangement providing an electromagnetic radiation in a visible spectrum range, an infrared spectrum range, or both spectrum ranges; a light transmission arrangement for channeling the electromagnetic radiation through, or reflected from, the blood of the patient; and one or more light detection arrangements are distributed around the blood of the patient, each of the plurality of light detection arrangements receiving portions of the channeled electromagnetic radiation from the light transmission arrangement and capturing amplitudes of a frequency range of the channeled electromagnetic radiation, wherein when pixel data output value of the radiation exceeds a reference pixel data output value within any of the light detection arrangements, a clotting event has been detected. The system wherein the light detection arrangement, further comprises: a detector comprised of a plurality of pixels arranged in rows and columns on a planar surface; a lens to focus the channeled electromagnetic radiation onto the plurality of pixels, the radiation incident substantially perpendicular to the planar surface. The system wherein an anti-coagulant is injected into the blood of the patient. The system wherein the computation device arrangement, further comprises: a memory to store a plurality of pixel data output values; and a computation device arrangement computing spatial, temporal, or spatial and temporal analysis on the captured amplitudes corresponding to pixel data output value of the light detection arrangement, a comparator to compare a reference pixel data output value and the channeled electromagnetic radiation of the specimen, wherein hardware to perform the comparator operation is selected from the group consisting of a field programmable gate array (FPGA), a multi-core central processing unit (MC-CPU), a graphics processing unit (GPU), and a machine learning (ML) device. The system wherein each of the light detection arrangements detecting blood clots to a depth of at least of 10 mm. The system wherein the blood of the patient being measured flows within either a cannula of an extracorporeal blood circulation system, a vein of the patient, or an artery of the patient.

In another embodiment, a method for detecting and correcting a formation of a blood clot in blood of a patient comprising the steps of: providing an electromagnetic radiation source that generates light in a visible spectrum range, an infrared spectrum range, or both spectrum ranges; channeling the light through, or reflected from, the blood of the patient; capturing amplitudes over a frequency range of the channeled electromagnetic radiation; computing spatial, temporal, or spatial and temporal analysis on the captured amplitudes corresponding to pixel data output value of the light detection arrangement, wherein when the pixel data output value reaches a reference pixel data output value, an anti-coagulant is injected into the blood of the patient. The method further comprising the steps of: arranging a plurality of pixels in rows and columns on a planar surface; focusing the channeled electromagnetic radiation onto the plurality of pixels using a lens, the radiation incident substantially perpendicular to the planar surface, wherein each of the plurality of pixels comprises at least a red, a green, and a blue sub-pixel, the pixel data output of the light detection arrangement, at a minimum, only requires the response corresponding to the output of the red sub-pixel (wavelengths 625-740 nanometers) to detect blood clotting. The method further comprising the steps of: storing a plurality of pixel data output values into a memory; and comparing a reference pixel data output value and the channeled electromagnetic radiation of the specimen to detect a clotting event, wherein hardware to perform the comparator operation is selected from the group consisting of a field programmable gate array (FPGA), a multi-core central processing unit (MC-CPU), a graphics processing unit (GPU), and a machine learning (ML) device. The method wherein the blood of the patient being measured flows within either a cannula of an extracorporeal blood circulation system, a vein of the patient, or an artery of the patient.

In another embodiment, a system for a measurement of clot formation in blood comprising: at least one light source arrangement providing an electromagnetic radiation bandwidth operating in a visible spectrum range, an infrared spectrum range, or both spectrum ranges; a light transmission arrangement for channeling the electromagnetic radiation through, or reflected from, the blood; one or more light detection arrangements receiving the channeled electromagnetic radiation from the light transmission arrangement to capture amplitudes over a frequency range of the channeled electromagnetic radiation; and a computation device arrangement computing spatial, temporal, or spatial and temporal analysis on the captured amplitudes corresponding to a pixel data output value of the light detection arrangement, wherein when the pixel data output value exceeds a reference pixel data output value, an action is performed. The system wherein the action that is performed is selected from the group consisting of injecting an anti-coagulant, changing the blood flow rate, raising a flag, issuing an alarm, raising temperature, and lowering temperature. The system wherein two or more light detection arrangements are positioned around a volume of the blood to collect the channeled electromagnetic radiation, each capable of detecting a clotting event, the two or more of the light detection arrangements each receives a different component of the channeled electromagnetic radiation. The system wherein the blood being measured flows within either a cannula of an extracorporeal blood circulation system coupled to a patient, a vein of the patient, or an artery of the patient. The system wherein hardware for the computation device arrangement is selected from the group consisting of a field programmable gate array (FPGA), a multi-core central processing unit (MC-CPU), a graphics processing unit (GPU), and a machine learning (ML) device. The system wherein the light detection arrangement, further comprises: a detector comprised of a plurality of pixels arranged in rows and columns on a planar surface; and a lens to focus the channeled electromagnetic radiation onto the plurality of pixels, the radiation incident substantially perpendicular to the planar surface, wherein each pixel is subdivided into a red, a green, and a blue sub-pixel, and blood clotting can be detected by using the red sub-pixel data of the pixel data output value from the light detection arrangement.

In another embodiment, a system for a measurement of clot formation in blood of a patient comprising: at least one light source arrangement providing an electromagnetic radiation bandwidth operating in a visible spectrum range, an infrared spectrum range, or both spectrum ranges; a light transmission arrangement for channeling the electromagnetic radiation through, or reflected from, the blood of the patient; and two or more light detection arrangements are positioned around a volume of the blood to collect the channeled electromagnetic radiation, each capable of detecting a clotting event, the two or more of the light detection arrangements each receives a different component of the channeled electromagnetic radiation from the light transmission arrangement and capturing amplitudes within a frequency range of the channeled electromagnetic radiation, wherein when a pixel data output value of the radiation exceeds a reference pixel data output value within any of the light detection arrangements, a clotting event has been detected. The system wherein once the clotting event has been detected, perform an action that is selected from the group consisting of injecting an anti-coagulant, changing the blood flow rate, raising a flag, issuing an alarm, raising temperature, and lowering temperature. The system wherein the blood being measured flows within either a cannula of an extracorporeal blood circulation system of the patient, a vein of the patient, or an artery of the patient. The system wherein hardware to perform the comparator operation is selected from the group consisting of a field programmable gate array (FPGA), a multi-core central processing unit (MC-CPU), a graphics processing unit (GPU), and a machine learning (ML) device. The system wherein the light detection arrangement, further comprises: a detector comprised of a plurality of pixels arranged in rows and columns on a planar surface; and a lens to focus the channeled electromagnetic radiation onto the plurality of pixels, the radiation incident substantially perpendicular to the planar surface. The system wherein the computation device arrangement, further comprises: a memory to store a plurality of pixel data output values; and a computation device arrangement computing spatial, temporal, or spatial and temporal analysis on the captured amplitudes corresponding to pixel data output value of the light detection arrangement, a comparator to compare a reference pixel data output value and the channeled electromagnetic radiation of the blood of the patient.

In another embodiment, a method for detecting and correcting a formation of a blood clot in blood comprising the steps of: providing at least one light source arrangement that provides an electromagnetic radiation bandwidth operating in a visible spectrum range, an infrared spectrum range, or both spectrum ranges; channeling the light through, or reflected from, the blood of the patient; capturing amplitudes over a frequency range of the channeled electromagnetic radiation; and computing spatial, temporal, or spatial and temporal analysis on the captured amplitudes corresponding to a pixel data output value of the light detection arrangement, wherein when the pixel data output value exceeds a reference pixel data output value, an action is performed. The method, wherein the action that is performed is selected from the group consisting of injecting an anti-coagulant, changing the blood flow rate, raising a flag, issuing an alarm, raising temperature, and lowering temperature. The method wherein two or more light detection arrangements are positioned around a volume of the blood to collect the channeled electromagnetic radiation, each capable of detecting a clotting event, the two or more of the light detection arrangements each receives a different component of the channeled electromagnetic radiation. The method wherein the blood of the patient being measured flows within either a cannula of an extracorporeal blood circulation system, a vein of the patient, or an artery of the patient. The method wherein hardware to computing spatial, temporal, or spatial and temporal analysis is selected from the group consisting of a field programmable gate array (FPGA), a multi-core central processing unit (MC-CPU), a graphics processing unit (GPU), and a machine learning (ML) device. The method further comprising the steps of arranging a plurality of pixels in rows and columns on a planar surface; and focusing the channeled electromagnetic radiation onto the plurality of pixels using a lens, the radiation incident substantially perpendicular to the planar surface, wherein each of the plurality of pixels comprises at least a red, a green, and a blue sub-pixel, the pixel data output of the light detection arrangement, at a minimum, only requires the response corresponding to the output of the red sub-pixel (wavelengths 625-740 nanometers) to detect blood clotting.

### BRIEF DESCRIPTION OF THE DRAWINGS

Please note that the drawings shown in this specification may not necessarily be drawn to scale and the relative dimensions of various elements in the diagrams are depicted schematically. The inventions presented here can be embodied in many different forms and should not be construed as limited to the embodiments set forth herein. Rather, these embodiments are provided so that this disclosure will be thorough and complete, and will fully convey the scope of the invention to those skilled in the art. In other instances, well-known structures and functions have not been shown or described in detail to avoid unnecessarily obscuring the description of the embodiment of the invention. Like numbers refer to like elements in the diagrams.

FIG. 1A depicts one embodiment of a static RGB Detector in accordance with the present disclosure.

FIG. 1B illustrates another embodiment of a static RGB Detector in accordance with the present disclosure.

FIG. 1C shows an embodiment of a dynamic RGB Detector in accordance with the present disclosure.

FIG. 1D presents a biological specimen coupled by blood flow to the embodiment presented in FIG. 1C in accordance with the present disclosure.

FIG. 1E depicts a block diagram of a blood clot sampling and corrective system in accordance with the present disclosure.

FIG. 2A shows the mechanical system used to measure coagulation in accordance with the present disclosure.

FIG. 2B presents a map of the clotting and reference sites in accordance with the present disclosure.

FIG. 2C illustrates a block diagram of the comparison system for FIG. 2A in accordance with the present disclosure.

FIG. 2D shows the RGB values of the reference site and the clotting site with a 2x diluted hemostatic agent in accordance with the present disclosure.

FIG. 2E depicts the RGB values of the reference site and the clotting site with blood depth of 5 mm in accordance with the present disclosure.

FIG. 2F presents the test results of the measured R (red) value in the reference sites and the clotting sites in accordance with the present disclosure.

FIG. 3A depicts the block diagram of the electronics used to measure coagulation in accordance with the present disclosure.

FIG. 3B illustrates a process flow of detecting and correcting for blood clots in accordance with the present disclosure.

FIG. 3C presents a block diagram of an ECMO system with anti-coagulant in accordance with the present disclosure.

FIG. 4 shows the RGB detector in relationship to the cannulas (tube) in accordance with the present disclosure.

FIG. 5A depicts the cross sectional view of one embodiment of flexible LED and flexible sensor arrangement in accordance with the present disclosure.

FIG. 5B illustrates the cross sectional view of another embodiment of multiple LED and multiple sensor arrangement in accordance with the present disclosure.

FIG. 5C presents the cross sectional view of an embodiment of an embedded LED and embedded sensor arrangement within the cannulas in accordance with the present disclosure.

FIG. 6 depicts the process flow of a self-correcting coagulant system in accordance with the present disclosure.

FIG. 7A presents a spatial view of a small blood clot within the field of view (FOV) in accordance with the present disclosure.

FIG. 7B illustrates the spatial view of the small blood clot a short time later within the field of view (FOV) in accordance with the present disclosure.

FIG. 7C depicts the spatial view of the small blood clot after another short time interval within the FOV in accordance with the present disclosure.

FIG. 8A illustrates a block diagram view of an embodiment of RGB database accessed temporally and spatially in accordance with the present disclosure.

FIG. 8B presents a block diagram view of another embodiment of an multi-time-space/computation blood clotting machine in accordance with the present disclosure.

FIG. 9 depicts a flow chart the RGB Detector using machine learning in accordance with the present disclosure.

FIG. 10 presents a block diagram of a machine training/learning system in accordance with the present disclosure.

### DETAILED DESCRIPTION OF THE INVENTION

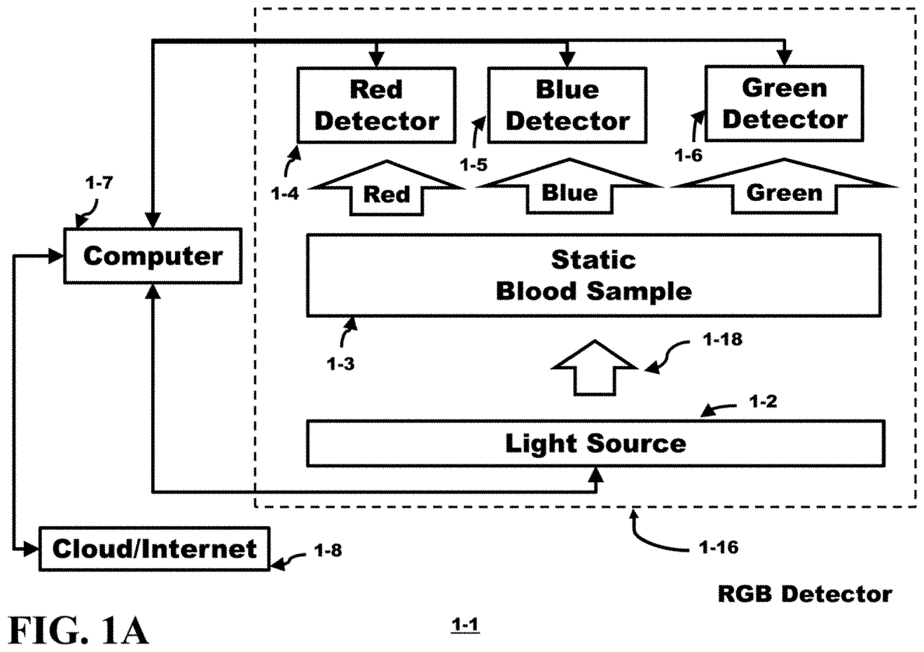

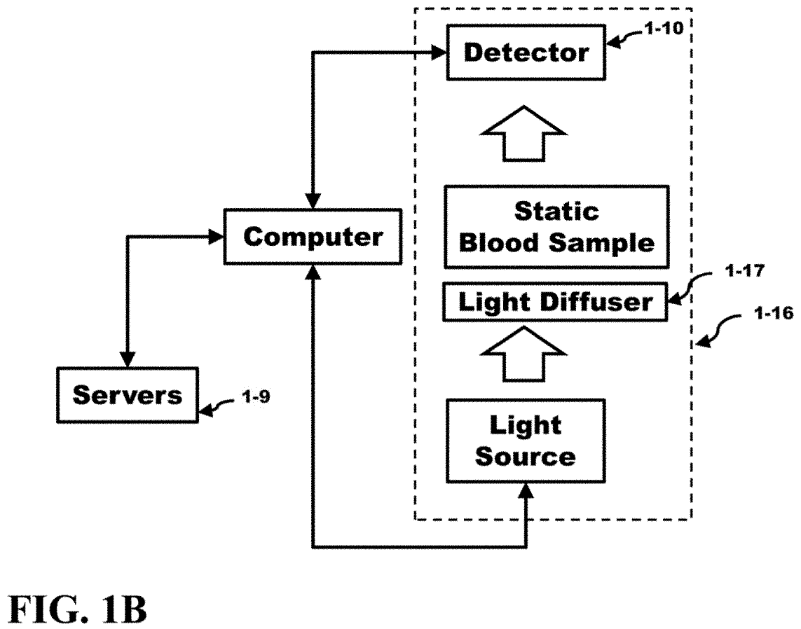

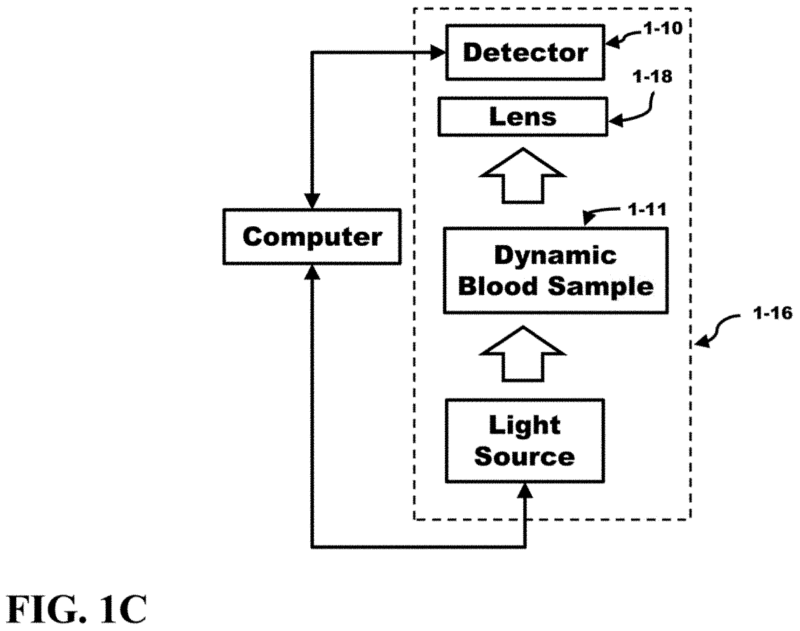

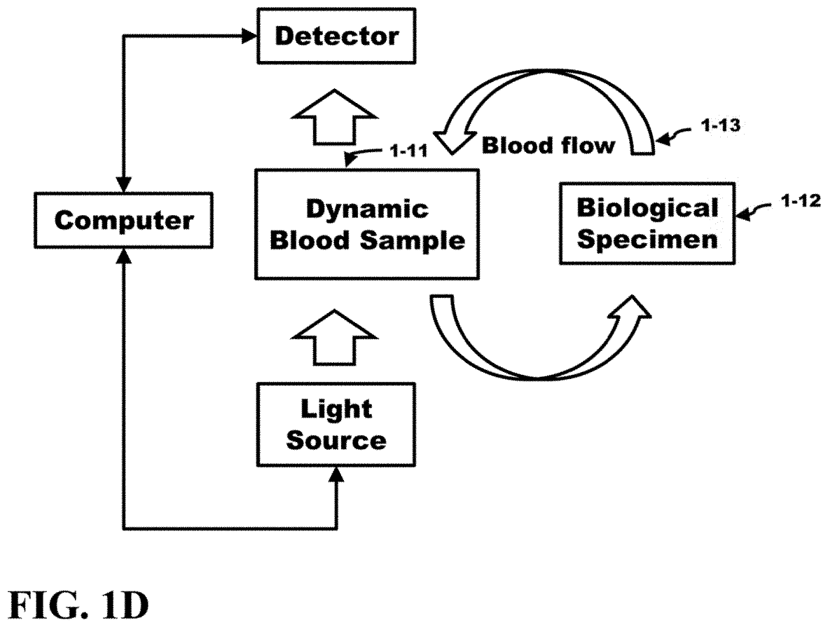

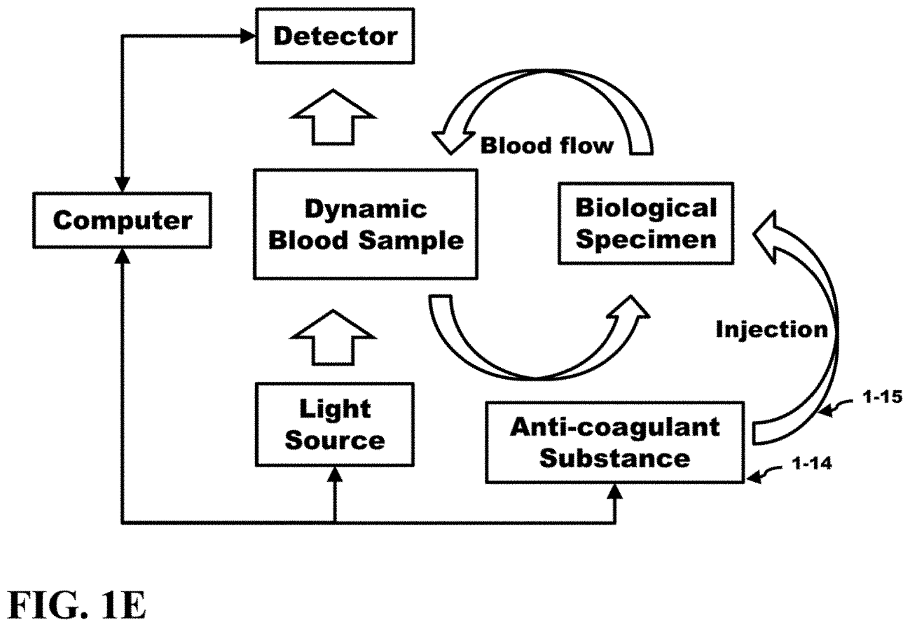

FIG. 1A illustrates a system 1-1 that transmits light 1-18 from a light source 1-2 emitting white light through a static blood sample 1-3 where static implies stationary blood. Separate color detectors (Red, Blue, and Green) are used to measure each of the three primary colors after the sample has been illuminated by the light source. The detectors 1-4 to 1-6 can provide information on a spatial, a temporal, or a combination of a spatial and a temporal matrix pattern. A first blood sample is measured and stored in memory as the reference sample. A second blood sample is introduced and then measured, but first, a coagulant is added to the second blood sample. A series of timed images are captured by the detectors and stored into memory. These timed images provide information on the condition of the blood. For example, at a frame rate of 750 fps, an image is captured every 1.3 ms. Each of these images can be compared to the reference to determine the condition of the blood. If the third image captured at 2.6 ms indicates that no significant difference in transmissivity exists when compared to the reference slide, then any effects of the coagulant on the blood sample have not been detected yet. However, after a quarter million frames (˜300 s), the transmissivity changed from 130 units to about 80 units. The extracted color information is provided to the computer 1-7 which can analyze the results using different algorithms. The spatial and temporal algorithms can perform moving averages, moving average over time, moving averages over space, and processes and techniques similar to those used in radar processing. All of the data can be stored in memory and the accumulated databases can be shared with the Internet or stored in the Cloud 1-8. The computer can also offload any algorithmic computations onto the Cloud, as well. The components within the dotted box 1-16: light source 1-2, blood sample 1-3, and the detectors 1-4 to 1-6 comprise an RGB Detector.

FIG. 1B depicts another embodiment of the RGB Detector 1-16 with a detector 1-10 and light diffuser 1-17. The light diffuser spreads out and diffuses the light preventing the camera from being saturated when there is too much light. The detector can be an area array of light sensors. The light sensors are arranged in rows and columns. For example, a CCD camera or a CMOS camera can be used as detectors. These cameras are fabricated in one of many semi-conductor processing lines, the pixels are arranged on the planar surface of the processed semi-conductor chip. Each x-y array pixel in these cameras is comprised of at least three different sub-pixels. In one embodiment, the first sub-pixel captures the R (red) color, the second sub-pixel captures the G (green) color, and the last sub-pixel captures the B (blue) color. Other sub-pixel combinations are used; some configurations depend on the manufacturer. The measured output of the detector is comprised of results of the three sub-pixels. The results of the RGB can be segregated from each other and analyzed independently or they can be combined in different proportions and analyzed together. The inventors have discovered that the R (red) color is highly correlated to the blood clotting event. The other two primary colors: G (green) and B (blue) show reduced response to the blood becoming clotted. However, the large changes to the R results correlate with the measured blood clotting events. In one embodiment, only the R pixels are sampled, viewed, and analyzed to make an assessment of the amount of blood coagulation or blood clotting that has occurred.

FIG. 1C shows another embodiment of the RGB Detector 1-16. The blood sample 1-11 is now dynamic and its image is focused using a lens 1-18. The focused image is captured by pixels within the array of the detector. The blood sample images comprise the blood while the blood is moving. In one embodiment, the blood is being circulated in a loop within the system. For example, in ECMO, the cannula, being transparent, visibly shows the moving blood stream. The blood stream is actively monitored for any clotting events. New samples of blood arrive continuously within the cannula. A computer adjusts the intensity of the light source and the detector registers the RGB color content instantaneously. The R detector (corresponding to the red pixel) is very sensitive to any clotting events forming in the stream. The change in the R color's transmissivity as a function of clotting events can be used to quickly identify blood coagulation.

FIG. 1D presents a biological specimen 1-12 coupled via blood flow 1-13 to the dynamic blood sample 1-11. The specimen's blood flow is monitored by the detector. In particular, the R reading is monitored to note if any coagulation is occurring. The system in FIG. 1D can be used to identify clotting events in a patient's blood flow. Once the R reading exceeds a pre-defined threshold level, the system is defined as experiencing blood clots.

FIG. 1E illustrates a detection and correction system to maintain the blood clot levels within a given tolerance range. The blood flow of the biological specimen is constantly monitored. The detector measures to determine when a pre-defined threshold level is exceeded. This indicates the onset of a blood clot. Once the blood clot has been detected, the computer applies a signal to the anti-coagulant substance 1-14 which injects 1-15 a set amount of anti-coagulant such as Heparin, xarelto, pradaxa, eliquis, lixiana, etc. into the blood stream. The anti-coagulant can be introduced into the patient through a newly formed intravenous port or use an intravenous port already in existence. After a short time period, the blood is monitored again. If the detector continues to measure an exceeded threshold level, inject another set amount of anti-coagulant and repeat the test. If the detector measures a value below the threshold value, the blood clotting events has been controlled. The system continues to monitor the blood flow for blood clot formation. Another embodiment of the system can include a comparison to decide if the clotting value is approaching one or more alarms. An upper threshold clotting level can be set to issue a critical alarm. This upper clotting level alarm indicates that the patient may be approaching death and imminent corrections are required.

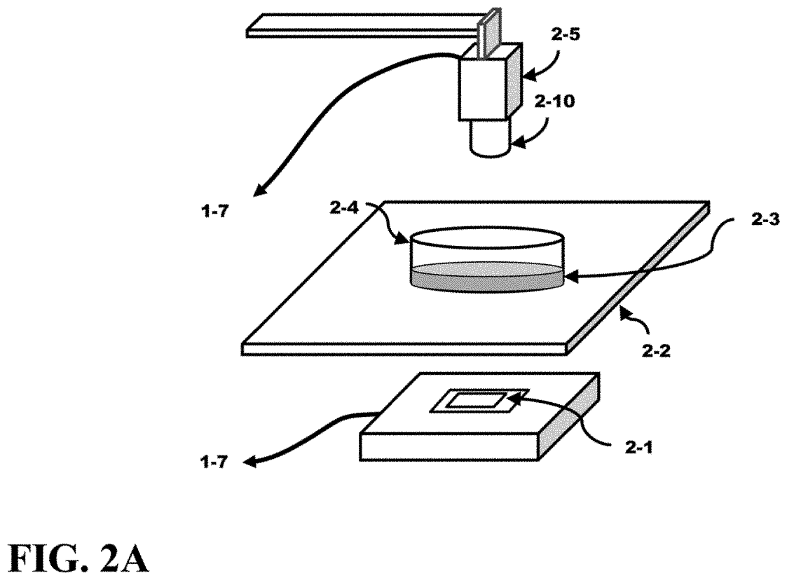

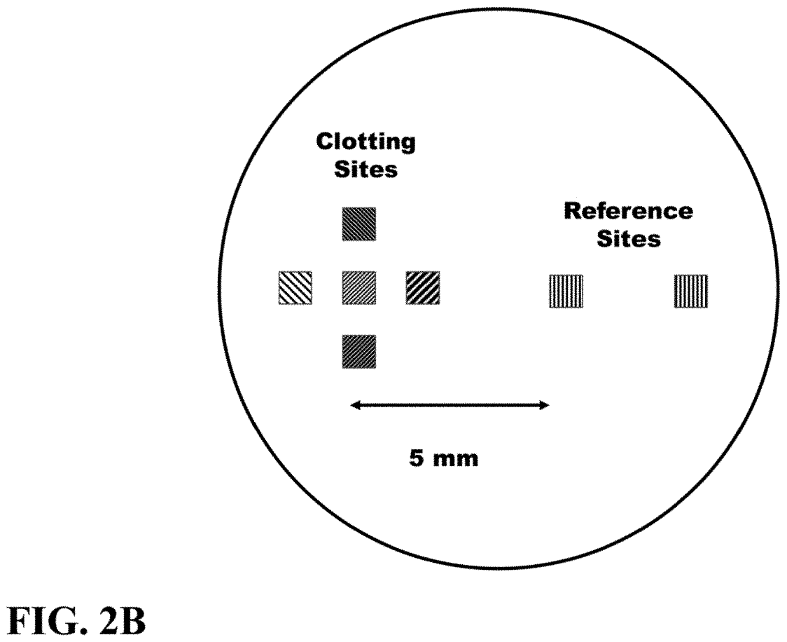

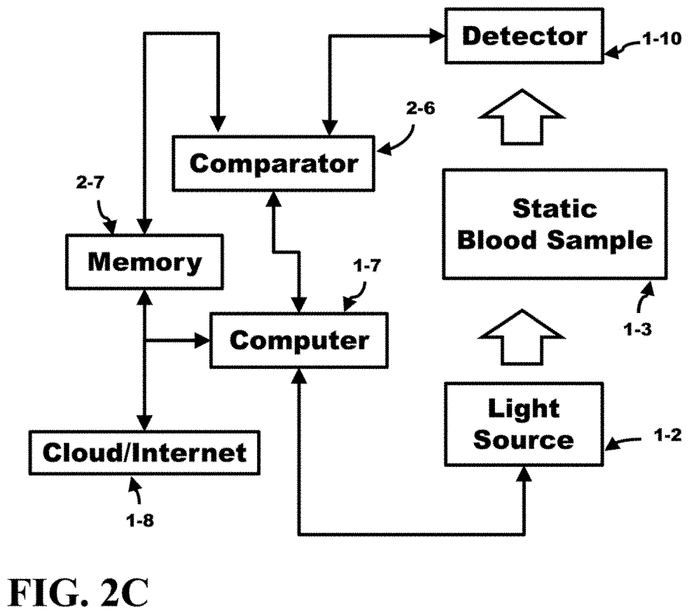

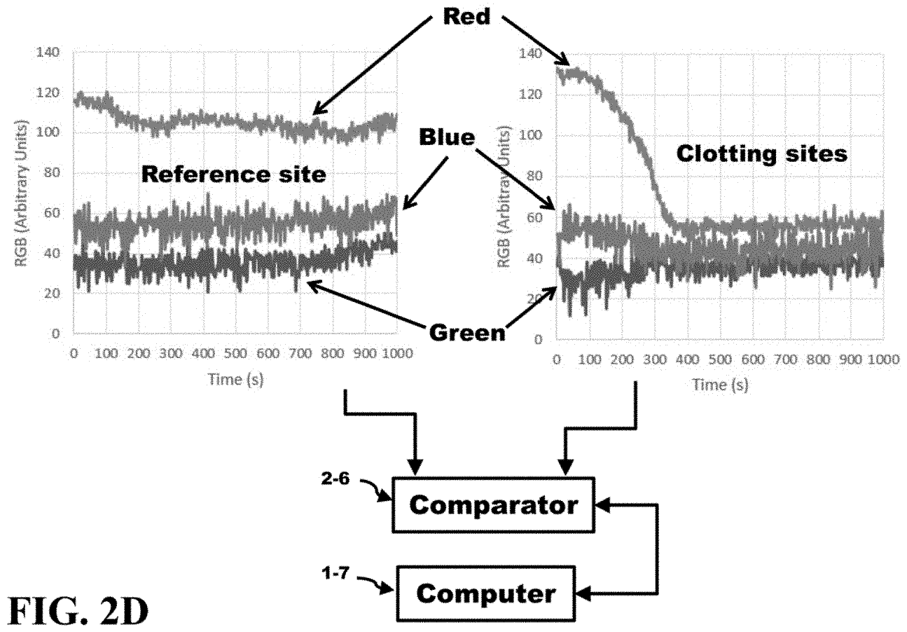

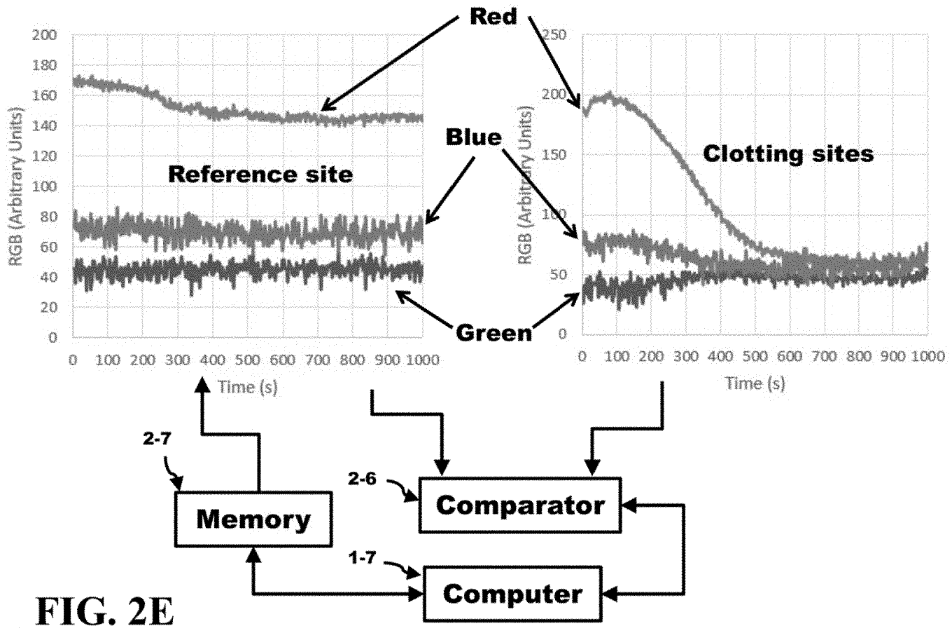

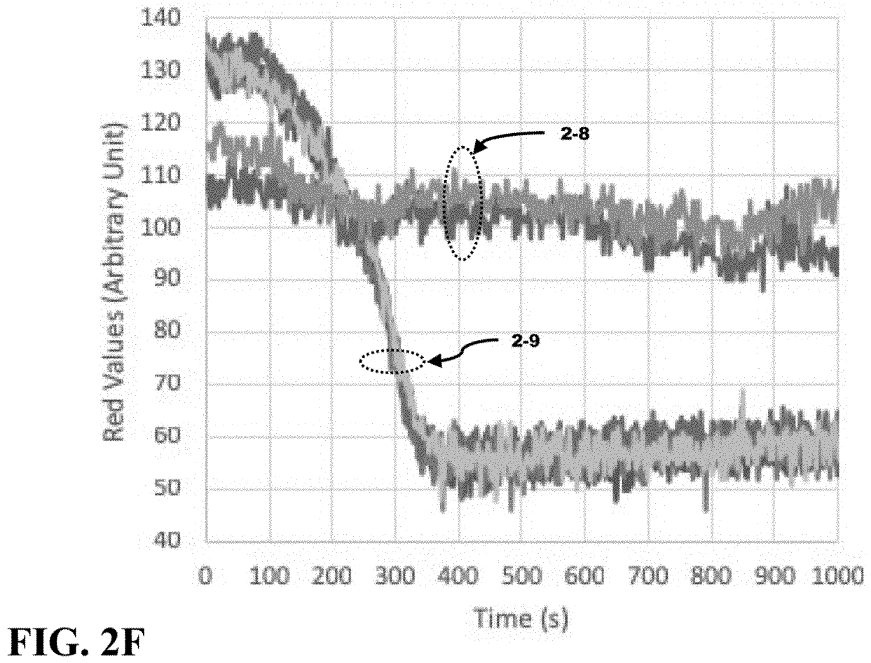

FIG. 2A depicts one embodiment of a static blood clotting detector apparatus used to obtain measurements using a high frame rate camera on blood during the coagulation event to determine the changes in red, green, and blue (RGB) values. Each of the pixels in the camera array is sub-divided into a plurality of sub-pixels. In one embodiment, the pixel is sub-divided into three primary colors: red (R), green (G), and blue (B) sub-pixels. To detect the onset of coagulation, these three sub-pixel properties can be captured by a high frame rate camera, which offer high resolutions and a wide dynamic range. The high frame rate (750 f/s) camera can measure the response of each of the three primary colors individually to the coagulation event. The blood flows through the tubes very rapidly—up to several centimeters per second. To detect a small blood clot that is quickly passing through the field of view requires a detector (camera) having a high frame rate image capturing capability. At least two benefits occur by collecting the plurality of measured results. First, the data can be used to reduce the noise by using various techniques; such as averaging, and strengthen the signal, thereby improving the signal to noise ratio and overall measurements. Second, the data provides samples for a global database that can be used in improving the machine learning systems.

The apparatus includes a camera 2-5 and optic lens 2-10. The camera's output is provided to a computer 1-7. The camera is a Basler model number acA640-750 um which can take up to 750 frames per second. The sensor area of the CMOS camera is 3.1 mm×2.3 mm while the pixel area occupies an area of 4.8 μm×4.8 μm. An Edmund Optics Lens 59870 is used to focus the light. It has a Field of View (FoV) of 61.4 mm and a max sensor format of 30.9°. Lampire Laboratories provided the sheep's blood 2-3 placed in the reflector tube 2-4, having a sodium heparin content of 1000 units/mL and a shelf life of ˜10 days at 4° C. The heparinized blood makes it possible to control the coagulation time and obtain results under various conditions (as explained below). This checks the consistency of the experiments. A white LED 2-1 provides the source lighting whose intensity can be controlled by a computer 1-7. A light diffuser 2-2 diffuses the light from the LED to avoid saturating the camera while the reflector tube helps to show greater blood depths.

Shown in FIG. 2B are five clotting sites within 250 μm of each other and two reference sites that are 5 mm and 7.5 mm away from the clotting sites. For our experiments, a drop of the hemostatic agent is added to the blood samples in the five left clotting sites. The camera measures, in real time, the RGB values of the pixels in these sites. A reference site about 100 pixels (550 μm) away is also measured using the RGB values. To reduce variations, an average of the clotting value is taken over 100 samples every 2 milliseconds.

FIG. 2C illustrates one embodiment of the test setup that was used. To start the coagulation, a Frenna hemostatic agent which has a 25% aluminum chloride content is added to the clotting site samples. Other clotting agents, such as Celox, have a granular form, which proved to have poor precision as compared to the Frena solution. The blood sample 1-3 is illuminated from below by a white 30-W LED light source 1-2.

In the one of the embodiments of the system, a compact optical device comprising the light source and detector will be designed to conserve space. The device can be miniaturized to a point where the device can be inserted into the cannula or into the patient's blood vessels.

The light passes through the static blood sample 1-3 and the altered Light is captured by the RGB pixels of the detector 1-10 as a clotted image. The altered light, or ‘channeled electromagnetic radiation’, is comprised of any transmitted components and any reflected components from the blood sample. A memory 2-7 holds the reference image of the reference site. This reference image is recalled from memory and compared in the comparator 2-6 against the clotted image from the detector. Software in the computer 1-7 or available in the Cloud/Internet 1-8 analyzes the results of the comparison and decides where the clotting value stands with regard to the threshold clotting level alarm. The clotting level alarm indicates that the patient has exceeded the allowable threshold level and that a blood clot formation has been found indicating that some action is required.

The system was tested in five conditions: with hemostatic agent without dilution, with 2x dilution, and with 4x dilution, as well as greater blood depths of 5 mm and 10 mm. In each case, the RGB values for both the clotting site and reference site were collected. Two sets of the comparative results are plotted in FIG. 2D and FIG. 2E. The vertical scale has a maximum of 255. The clotting agent is added at time zero, and the RGB values are captured for the next 1000 seconds. The objective was to see if there exists a clear and distinct difference between the clotting and reference sites in terms of red, green, and blue values. For greater blood depths, the light diffuser and reflector tube are adjusted to illuminate the blood optimally. As a result, the maximum red value varies to some extent across the measured results.

FIG. 2D presents measured results of a reference site and a clotting site after a 2x diluted hemostatic agent was added to the clotting site. Note that the Green and Blue results show little or no difference between the two cases. As illustrated in the lower part of the figure, the actual image of the reference site and the actual image of the clotting site are applied to the comparator 2-6. The computer 1-7 or Cloud (not shown) compares the two results and determines the clotting level based on the threshold value.

FIG. 2E illustrates another set of measurements. The results of the reference site and the clotting site after the light penetrated through 5 mm of blood. Note that the Green and Blue results show little or no difference between the two cases. The Red (R) case does show a difference. As illustrated in the lower part of the figure, the stored image of the reference site is extracted from memory 2-7 and the actual image of the clotting site are applied simultaneously to the comparator 2-6. The computer 1-7 or Cloud (not shown) compares the two results and determines the clotting level based on the threshold value.

Even in the scenario of 10 mm of blood depth, where the difference in red values between the clotting and reference sites was comparatively small, there was a contrast of around 40%. This depth was chosen to simulate the conditions of ECMO, where the tube radius is around 10 mm. This contrast can be improved by using a brighter LED or adding a lens between the LED and the diffuser.

Although only two sets of results were presented, data was monitored and collected from other sites as well. The additional tests conducted included using an undiluted hemostatic agent, a 4x diluted hemostatic agent, and blood depth tests of 10 mm. In all of these measurements, the observation was that the red value is a consistent indicator of coagulation and drops far more than the blue and green values. As can be observed in the graphs above, the time taken for the clotting site red value to drop is proportional to the dilution factor of the hemostatic agent. More importantly, the red in the clotting site undergoes a more significant change than that of the reference site. Results have shown that the transmissivity of the primary red color shows a strong relationship to the formation of blot clots. The transmissivity of the other two primary colors (green and blue), comparatively speaking, change little during the coagulation event.

FIG. 2F plots only the red values for the clotting and reference sites illustrated in FIG. 2B. The variation is about five percent for clotting sites and about seven percent for reference sites before the passage of 950 seconds. The two reference sites 2-8 have relatively constant red values of about 105. Note that the red values of the 5 clotting sites 2-9 experience a dramatic change in the red value starting at about 130 at zero time and ending at about 60 at about 350 seconds.

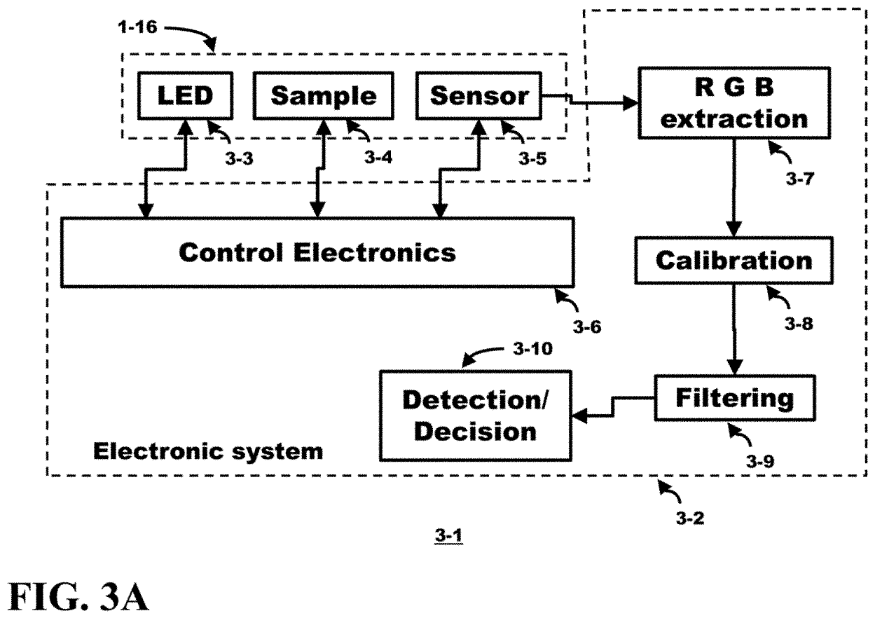

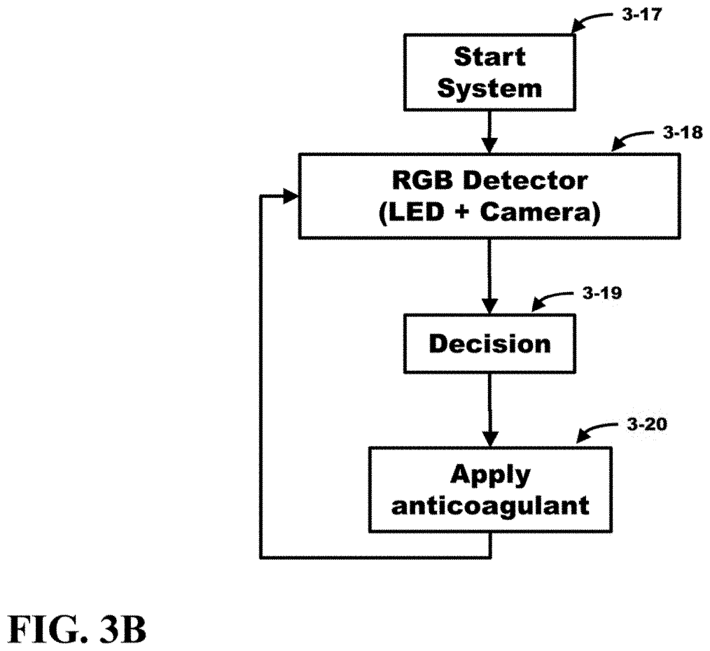

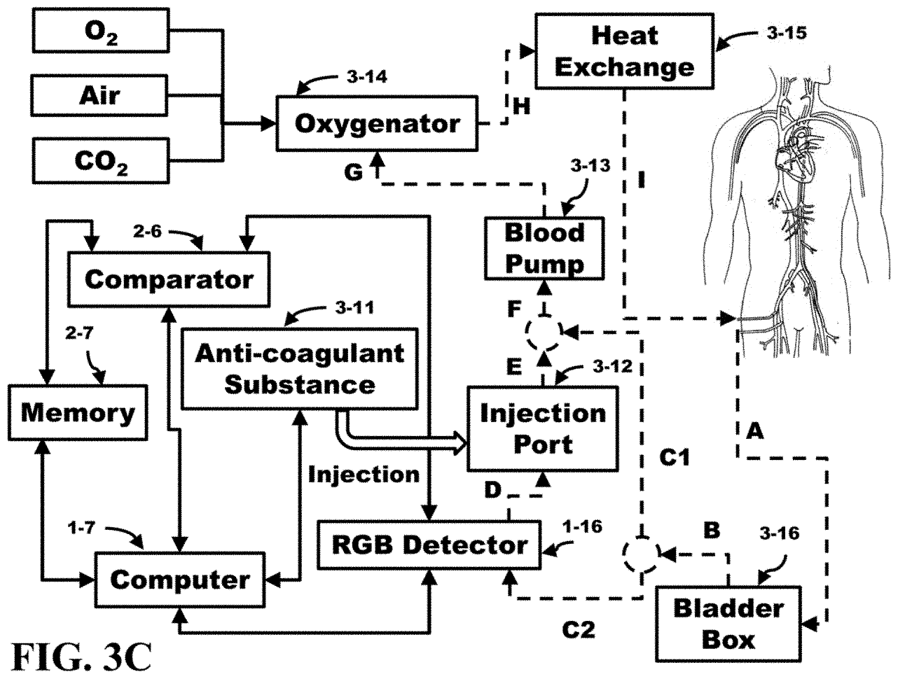

FIG. 3A illustrates the electronic block diagram 3-1 of the clot detecting system. The three components: the LED 3-3, the transmitted light after passing through the Blood Sample 3-4, and the Sensor 3-5 detecting the transmitted light make another embodiment of the RGB detector 1-16. The electronic system 3-2 comprises the control electronics 3-6 which activates, coordinates, and captures the data created between the LED and the sensor of the RGB detector. A flow chart illustrates the sensor 3-5 providing data to the R G B extraction block 3-7. The primary colors are extracted as a function of time and/or a function of position within the pixel array. The results are calibrated 3-8 against a reference. The reference could be a live image or a stored image as described earlier.

After calibration, the signal is filtered 3-9 to extract out the occurrence of coagulation from a space/time matrix result. The detection/decision 3-10 evaluates the filtering result and can provide the user with data about the size, position, and/or velocity of the blood clots. For example, in the comparison of one x-y array image against the reference image, various areas are darkened out indicating the locations of clots. Once the clots have been identified, each can be measured for location, width, height, closest neighbor, etc. Between additional timed measurements, the captured data can be used to calculate clot size, clot grouping size, clot velocity, etc. In one embodiment, the width of the detector spans the diameter of the cannula carrying the flow of blood.

A closed blood loop anti-coagulant system using the clot detecting system of FIG. 3A is depicted in the flowchart of FIG. 3B. The process starts 3-17, the RGB detector 3-18 comprising the light source (LED) and video recording source (Camera) continuously monitor the red, green, blue values of the transmitted light through the blood within the aperture of view. Note that earlier measurement results favor the ‘red’ response as being the indicator of when blood clots occur.

The visible spectrum ranges from wavelengths from about 380 (violet) to 740 (red) nanometers. Approximately, Red occupies the wavelengths 625-740 nanometers, Green occupies 500 to 565 nanometers, and Blue occupies 450 to 485 nanometers. The measured results indicate that the Red response experiences the largest change in its transmissivity through the blood sample. These changes occur at the Red wavelengths of 625-740 nanometers. By monitoring the Red wavelength bandwidth of transmitted light through blood samples, comparative measurements against a reference can be performed to detect blood clot formations.

Similarly, the visible spectrum extends from frequencies of about 480 (red) to 680 (violet) terahertz. Approximately, Red frequencies in visible spectrum extend from 405-480 THz, the Green frequencies range from 530-600 THz, and the Blue frequencies range from 620-680 THz. A frequency range of the visible spectrum can be monitored, for example, the Red frequency range of 405-480 THz can be evaluated. By monitoring the Red frequency range of transmitted light through blood samples, comparative measurements against a reference can be performed to detect blood clot formations.

Once clotting has been detected, a decision 3-19 is necessary to check if the Red color exceeds the threshold value. If the threshold value is exceeded, apply anti-coagulant 3-20 that can comprise: heparin, xarelto, pradaxa, eliquis, lixiana, etc.

FIG. 3C depicts one embodiment of a closed loop RGB detector coupled to an ECMO that maintains the concentration of blood clots to remain in the vicinity of a pre-set threshold value. The ECMO system portion comprises the cannula A, the bladder box 3-16, the cannula B, the cannula Cl, cannula F, the blood pump 3-13, cannula G, the oxygenator 3-14, the cannula H, the heat exchange 3-15, the cannula I, and the patient (all dotted arrowed lines form a closed circular blood loop). Assume that the patient requires the ECMO system, for example, they are receiving a ‘new’ lung. Blood is extracted from the patient on cannula A and moves into the bladder box 3-16. The fill capacity of the bladder box enables the blood pump. If the box is empty, there is nothing to pump, so disable the blood pump. If the box is full, there is much to pump, so enable the blood pump and move the blood through the remainder of the system. The bladder box prevents an excess negative pressure from occurring at the inlet to the blood pump. Since the patient is receiving a new lung, the oxygenator 3-14 is behaving like a ‘lung’ by adding oxygen to the blood flow and removing carbon dioxide from the blood flow. The heat exchange 3-15 warms the blood to the correct body temperature. Finally, the oxygen rich blood is feed back to the patient using cannula I. Note the many access ports where an RGB Detector can be located via the many cannulas in the system.

The closed loop RGB detector/comparator is located in the lower left of FIG. 3C. Two of its components: the RGB Detector 1-16 and the injection port 3-12 have been inserted into the blood path between cannula B and cannula F. These two components are not required to be next to one another or need to be necessarily located between cannula B and cannula F. The RGB detector and the injection port can be located anywhere along the blood path where there is an accessible external cannula from which blood samples can be monitored or substances can be infused. The RGB detector monitors the clot formation in the blood flow and provides that information to the computer 1-7 and the comparator 2-6. The computer, the comparator, and the reference image from the memory 2-7 are used to decide what the clotting level currently is. If the blood clot level is less than the threshold level, do nothing. However, if the blood clot level is greater than the threshold level, activate the anti-coagulant substance 3-11 and inject some of this substance into the inject port 3-12 and into the blood flow. Continue monitoring the patient, wait till the anti-coagulant distributes in the patient, then follow the previous two steps.

The threshold level is set to detection of small clots of around a few hundred micrometers in size to determine the early detection of blood coagulation. To achieve consistent results, more than 40 experiments were performed in which the RGB data was collected and the system was refined. Similar experiment can be performed to detect smaller clots and larger clots and to use all this data to map out a threshold level versus clot size chart.

In critical operations, where the use of extracorporeal blood circulation system is required, the cannula is an external tube that is inserted into the patient's veins or arteries to provide an entry/exit blood port on the patient. Additional cannulas are used to couple external equipment together creating a blood loop between the entry/exit blood ports of the patient. These additional cannulas in the system (for example, see B, F, G, H, and I in FIG. 3C) provides easy external access to the blood flow of the patient. The easy access is for way to couple in both the injection port 3-12 and the RGB detector 1-16 into the blood loop. In one embodiment, the RGB Detector is coupled to a cannula while the injection port uses the patient's intravenous setup that already has been established for the patient's procedure.

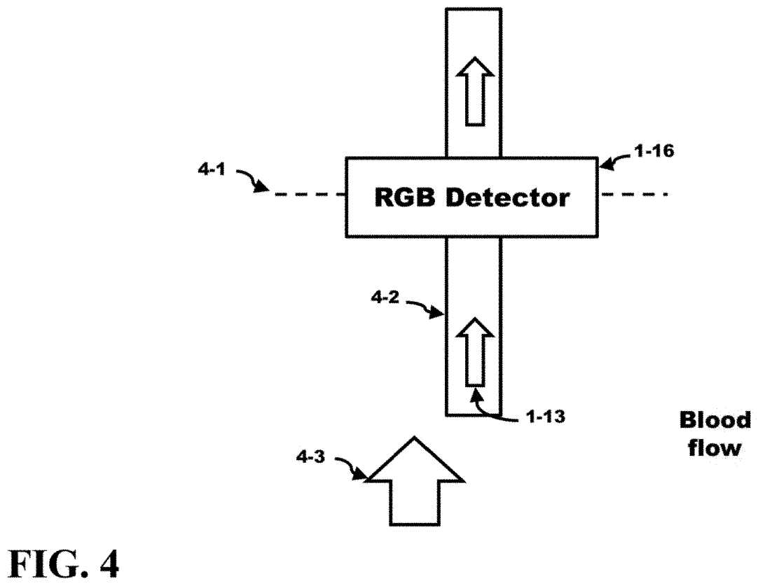

In another embodiment, the injection port and RGB detector are combined into one unit, such that, an ingress exists for the introduction of an anti-coagulant into the blood flow and the blood flow can be observed using the light source and detector. Such a structure is illustrated in FIG. 4. The cannula 4-2 carries a blood flow 1-13 and the cannula is coupled to an RGB Detector 1-16. The dotted line 4-1 indicates the location of the cross sectional view as seen from the perspective of the arrow 4-3.

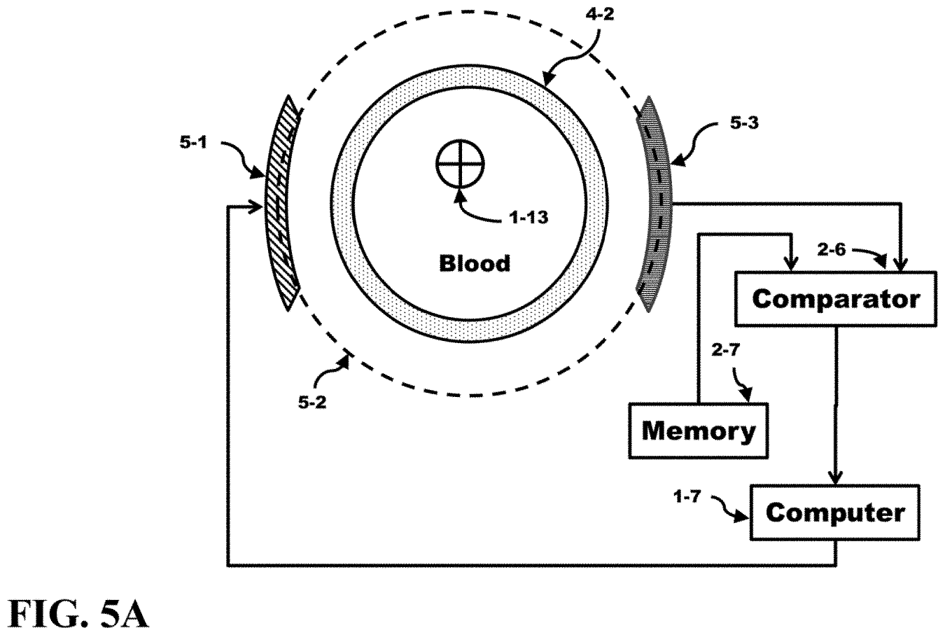

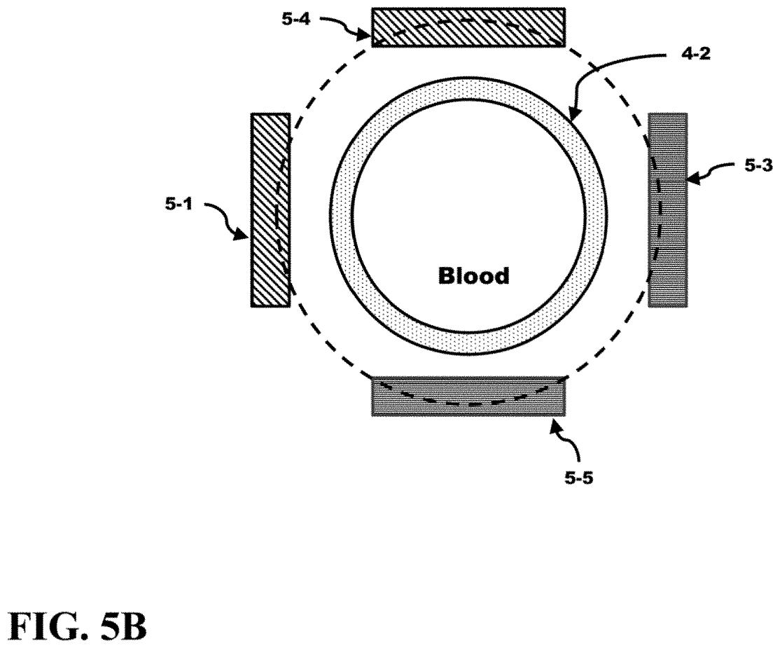

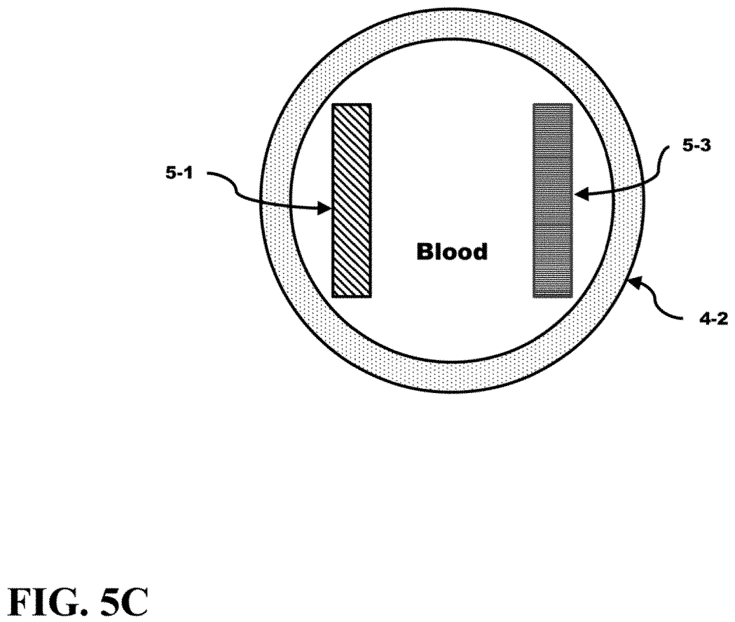

FIG. 5A-C illustrates several embodiments of these views along the dotted line 4-1 presented in FIG. 4 detailing the cross section between the LED, sensor, and the blood flow. The cannula 4-2 is a transparent tube that allows the transmission of visible light.

FIG. 5A illustrates a ring fixture 5-2 around the cannula and mounted on the ring fixture is a flexible LED 5-1 and flexible RGB light detector 5-3. The flexible material is a flexible plastic substrates that carries all required semiconductor electronics (LEDs, camera, amps, op amps, etc.) formed on a polyimide or a transparent conductive polyester film. In another embodiment, the flexible LED and RGB detector can be attached directly to the outside surface of the cannula (not shown) conserving space and removing the need for the ring fixture. Note that the detector and the LED are positioned on opposite out-sides of the cannula. The RGB detector can be a camera having some of the standard pixel sizes as used in industry. The tail of the arrow 1-13 indicates that the blood is flowing into the page. A wireless system to detect alarms and allow for additional controls to the system can be added to the detector.

The operation of FIG. 5A follows. As blood passes between the LED 5-1 and the sensor 5-3, light from the LED passes through the material of the cannula 4-2 and directly into the path of the blood flow. The transmitted light is altered after passing through the blood if the blood flow contains blood clots. The altered light then passes through the material on the opposite side of the cannula and into the sensor 5-3. The sensor analyzes the image for temporal and/or spatial information that may indicate the occurrence of blood coagulation. The comparator 2-6 compares the measured results with the reference extracted from the memory 2-7. The results are applied to the computer 1-7 which uses one of several algorithms to determine the characteristics of the one or more blood clots being analyzed. These characteristics include clot size, clot cluster size, clot position, clot size growth, clot cluster size growth, clot velocity, etc. Cluster size is a grouping of individual clots into tight clusters.

Another embodiment of the RGB electronic components combining with the cannula is presented in FIG. 5B. The ring fixture can be used to hold the components, or the components can be placed onto flexible material and then attached to the outside circumference of the cannula. The plurality (n) of LED-Sensors can be interleaved around the circumference of the cannula. FIG. 5B illustrates one case of where n=2. The light from LED 5-1 can be detected by the sensor at 5-3, the sensor 5-5, or both simultaneously. Similarly, the light from the second LED 5-4 can be detected by the sensor at 5-3, the sensor 5-5, or both simultaneously. One or more of the plurality of LEDs can be different than all the rest (red color, blue color, power, efficiency, etc.). One or more of the plurality of sensors can be different (technology types: CCD, CMOS).

Another embodiment of the RGB electronic components combining with the cannula is presented in FIG. 5C. The LED 5-1 and the sensor 5-3 are inserted directly into the cannula 4-2. In another embodiment, the flexible circuits can be used (not shown) to print the LED and sensor onto the inside surface of the cannula. Note that the detector and the LED are positioned on opposite in-sides of the cannula. In another embodiment, the flexible circuit may contain a wireless power supply. By applying a varying magnetic field outside the cannula, the wireless power supply generates a voltage to power the LED and sensor. The placement of the plurality of LEDs and sensors can also be interwoven along the inner circumference of the cannula. Algorithms within the computer can be used to collect the data from all the interwoven sensors, and use the data to extract information about the clot formation, its size, the number of members within a group, etc. An additional wireless system can be included to detect alarms. The wireless system can also allow for additional control capability to the system via a smart phone or tablet.

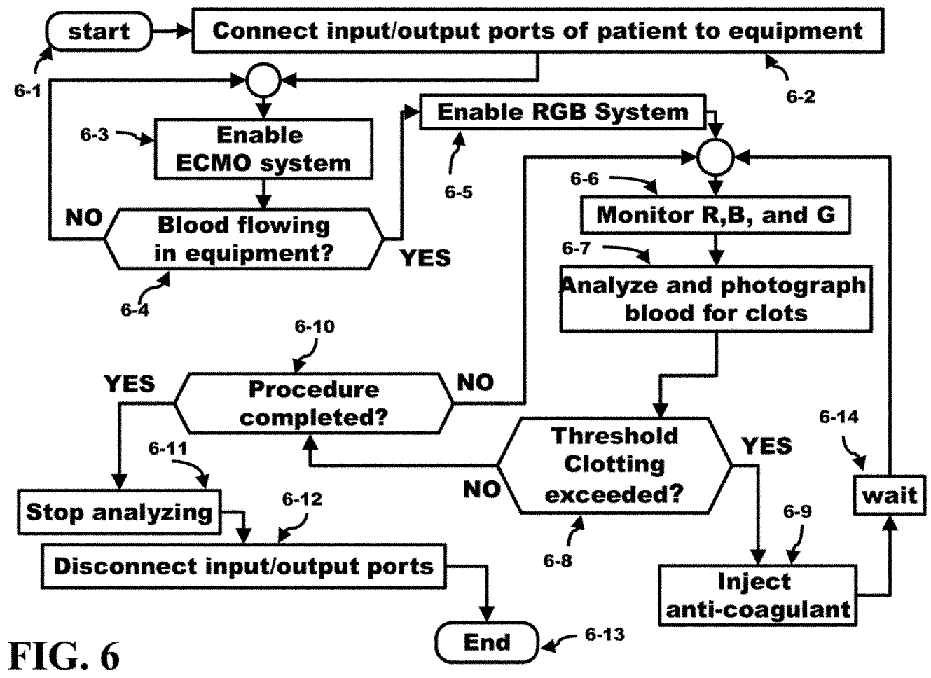

FIG. 6 presents a flow chart describing the operation of an ECMO controlled by a closed loop RGB detector to maintain the clot level at or below a specified threshold. At start 6-1, connect the patient's input/output ports to external equipment 6-2 using cannula. Next, enable the ECMO system 6-3. Once enabled, the blood will start flowing 6-4 in its closed blood loop. The blood loop comprises the output port, the bladder, the oxygenator, heater, the input port, and the internal veins or arteries. Once blood flows, enable the RGB system 6-5, then start monitoring the R, G, and B values 6-6. In some embodiments, only the R value is monitored. The image is photographed and analyzed for the occurrence of blood clots 6-7. Has clotting exceeded the threshold value 6-8? If yes, inject patient with anti-coagulant 6-9 such as, heparin, xarelto, pradaxa, eliquis, lixiana, etc. At wait 6-14, the patient waits till the anti-coagulant has distributed into its system, once complete, move to monitoring 6-6. However, if the threshold had not been exceeded, determine if the procedure is complete 6-10. If not, continue to monitor patient 6-6. Otherwise, if procedure is complete, stop analyzing 6-1, disconnect patient from equipment 6-12 and end 6-13.

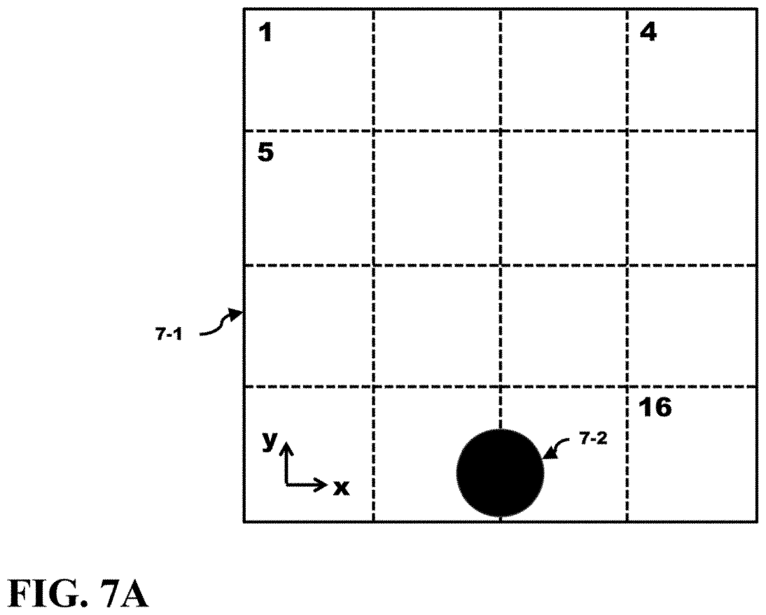

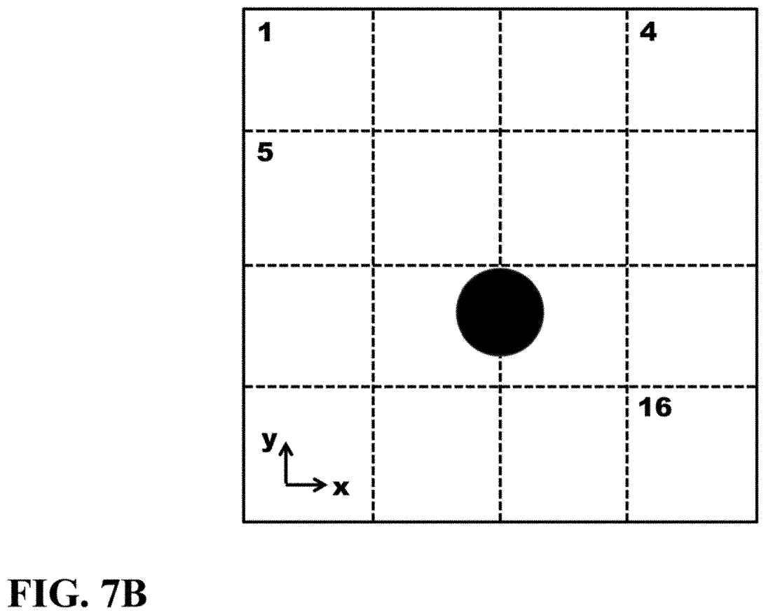

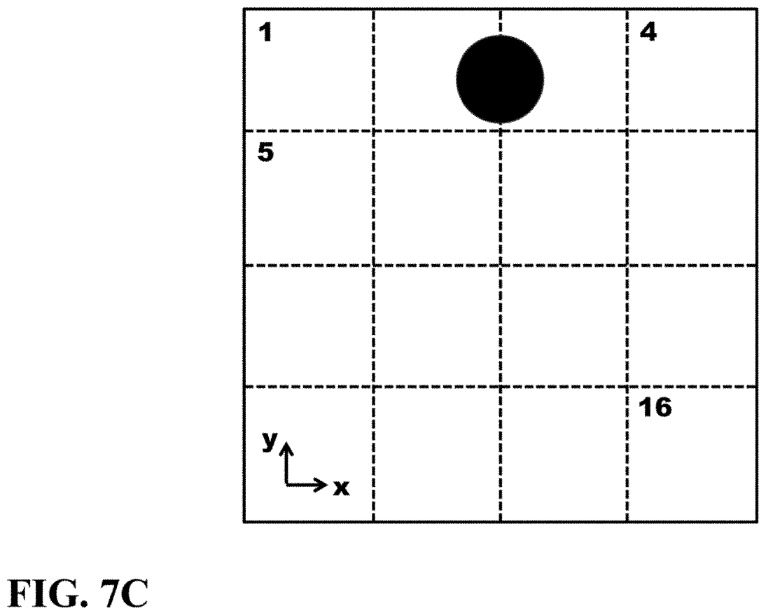

FIG. 7A-C depicts a blood clot 7-2 as it is changing in space and in time within an array 7-1. The array is a two dimensional pixel matrix representing the location of each pixel in the camera. Each pixel is further sub-divided into sub-pixels, where the sub-pixels comprise at least R, G, and B. The pixels in the array are numbered left to right, top to bottom, using Cardinal numbers: 1, 2, 3, . . . . For example, 4 is in the top left while 16 is in the bottom right. The 4×4 array is a very simple array, as there is a plurality of different pixel sized arrays. For example, the Basler camera used in the present experiments has a pixel array sized as 640 pixels×480 pixels. The accuracy of detection, measurement, and position of the clots improves as the array size is increased. The blood clot in FIG. 7A is located in pixels 14-15.

A threshold value is used in the system to identify a non-clotting patient from a clotting patient. The threshold level can be measured on each patient prior to the procedure and stored in a database. A threshold value is determined by the ratio of the area of the blood clot to the overall area of the total array. For example, the blood clot 7-2 had an area equal to about a ⅓ of a pixel then the threshold value would be equal to 1/48. Assume that this threshold value identifies the boundary between the patient clotting or not clotting. For any readings below 1/48, do nothing. For any reading above 1/48, inject an anti-coagulant.

FIG. 7B presents the blood clot at a first later time. The blood flow is in the positive y-direction and the blood clot moved about 1.25 pixels and can be found within pixels 10-11. FIG. 7C presents the blood clot at a second later time. The blood clot moved about 1.66 pixels and can be found within pixels 2-3.

The photos of FIGS. 7A-C can be translated into number of different databases. One database can include the pixel number and a Boolean result (True if clot in pixel, False if not). This database represents spatial and temporal pixel information. Various algorithms can be applied to the database to better understand the formation of the clot, the size of the clot, the movement of the clot, the structure of the clot, etc. As a though experiment for one possible algorithm, use the positions of the blood clot in FIGS. 7A-C with the following assumption: the sequences presented are equally spaced in time. Since the separation between the previous clot and the current clot is increasing, it appears that the fluid flow is experiencing an increase in acceleration. Working with the pixel locations and time, one can calculate velocities (pix/sec), interpolate positions as a function of time, etc.

One simple example of a spatiotemporal matched filtering method is a “delay and sum” approach. Consider the system in FIG. 7. The clot is moving in the y direction. Instead of making the decision (on the presence of the clot) based on a single frame (e.g. FIG. 7A), spatiotemporal matched filtering, in an example embodiment, could combine the signals from multiple frames after taking into account the delay and location shifts due to the movement of the clot along the y axis. The simple example assumes laminar flow of the particle and at a constant velocity, so knowing the time difference of the frames, we could apply the expected shifts in locations (here the pixels) before combining the signals. The shift is due to locational changes across time difference between frames.

In the embodiment above, assuming laminar flow and with constant known velocity (related to the flow rate), we can combine the signals from “pixels” 14 (and 15) in FIG. 7A with pixels 10 (and 11) in frame in FIG. 7B, and so on. After the combination of all respective pixels are done, a threshold for detection with multiple frames can be applied, which now has better SNR and lower false alarm probability. Constant False Alarm Rate (CFAR) techniques, similar to ones applied to radar detection, could be used to further enhance the detection method.

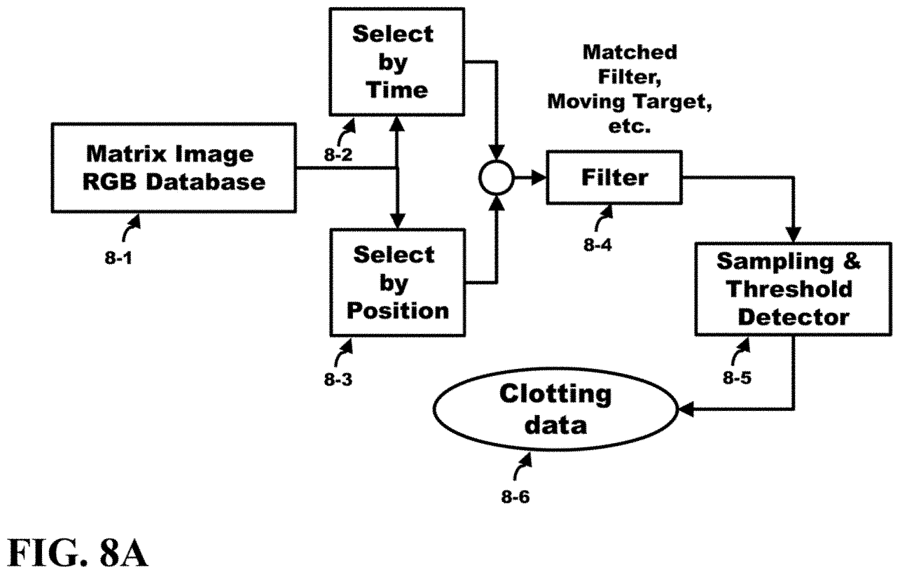

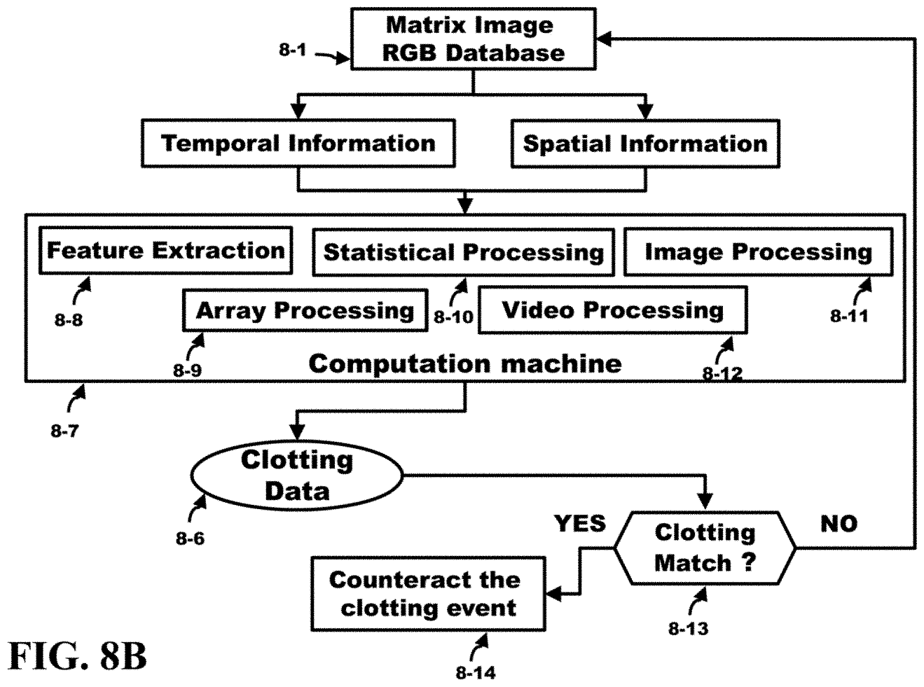

FIG. 8A presents a sequence using the matrix information and arriving at clotting data. The matrix image RGB database 8-1 includes stored and currently viewed images. The pixels can be selected by time 8-2 or by position 8-3 or by both simultaneously. Once the information is combined, the information is filtered. The filtering is a data processing manipulation that uses algorithms and provides results. For example, the data can be filtered in a ‘Matched Filter’ or a ‘Moving Target’ filter. The data is sampled, noise averaged, and compared in a threshold detector 8-5. The data that was sampled and measured comprises the clotting data 8-6 can be stored away. Then, in future searches, Machine Learning can use these databases to improve the overall system.

FIG. 8B depicts another embodiment of a computing machine that can detect and counteract clots events. The matrix image RGB database 8-1 includes stored and currently viewed images. The pixels can be selected temporally or spatially or both simultaneously. The information is then applied to a computation machine 8-7. The computation machine is a special propose machine that is designed to preform many multiplies and adds simultaneously. Some examples of the special purpose machines comprises: a field programmable gate array (FPGA), a multi-core central processing unit (MC-CPU), a graphics processing unit (GPU), and a machine learning (ML) device. The computation machine can use this hardware to perform feature extraction 8-8, statistical processing 8-10, image processing 8-11, array processing 8-9 and video processing 8-12. The result of these calculations creates a database of the clotting data 8-6. The database can be used to determine if a clotting match occurs 8-13. If there is no match, redo the process again. If a match occurred, counteract the clotting event by adding an anti-coagulant 8-14 to reduce the clotting event.

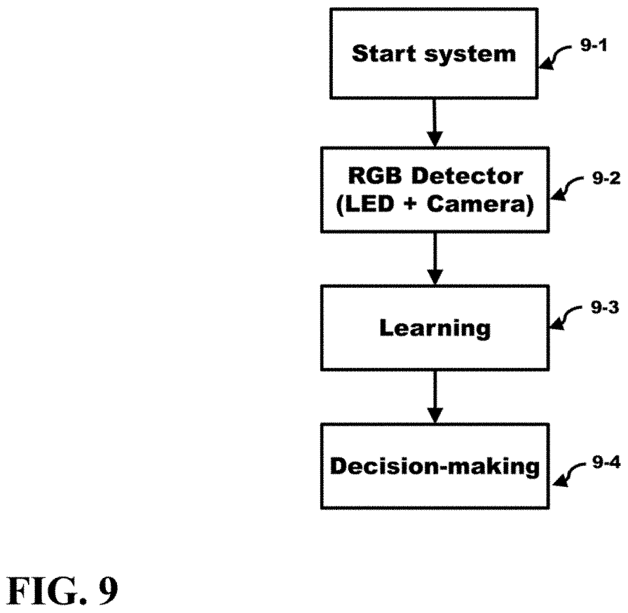

FIG. 9 illustrates a blood clotting system that uses machine learning to make decisions of blood clotting events. Once the system starts 9-1, the RGB detector 9-2 enables the light source and camera to collect images. These images may be used immediately or stored in memory and recalled from memory at a later date. The images are collected to form a collection. The collection is a large database that the user or others can add their images to and use. This collection forms the basis of learning 9-3 where the data reveals the characteristics of a blood clot. That knowledge is used in the decision making 9-4 step to reduce the clotting event.

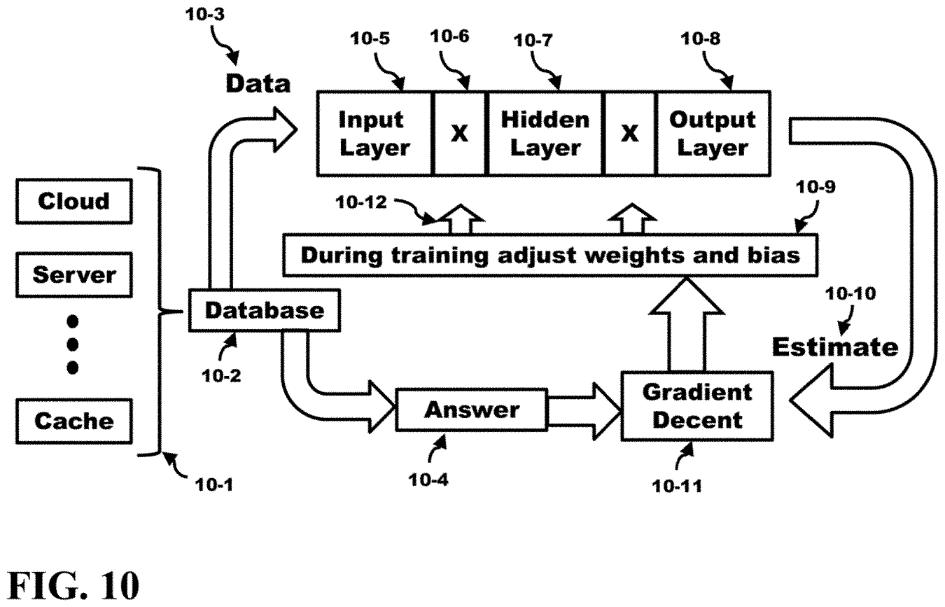

FIG. 10 shows a Supervisory Machine Learning (ML) Block diagram that uses the Gradient Decent 10-11 to adjust the weights and bias 10-9. A successful ML project requires the manipulation of large databases. The ML device performs specific tasks without proving explicit instructions. The ML device uses the massive database to help it to ‘learn’ what is needed. The databases 10-2 obtains its data from local cache, on-chip RAM, tape, disc, Memory array, server, Cloud, Internet 10-1. The database comprises data to train the network and more data to test the network. In supervisory learning, data and the answer is provided to the ML device. In one embodiment, the answer is a Boolean (True or False) and represents if the pixel is seeing a clot or not. So, for example, if the answer is True, data 10-3 representing a clot is applied to the input layer 10-5. Boolean implies that this is ‘classification’ problem (fraud: Yes or No; Spam Mail: True or False; Clot: True or False). During training, the weights and bias 10-9 change their values 10-12 and are continually applied and updated for each new input value applied to the neural network. The neural network comprises the input layer 10-5, the interconnect (with weights and bias), hidden layers 10-7 (one or more), another interconnect (with weights and bias), and an output layer 10-8. As time passes, the neural network makes a better and better estimate 10-10 that is compared to the answer 10-4 while the system is performing the gradient decent 10-11 and training the network.

A neural network (NN) is an adjustable-weighted network used with machine learning algorithms that can be used to classify inputs based on a previous training process. In a first embodiment of clot detection, a neural network is first trained on a first set of clotted and non-clotted images to detect clotting. Once the NN has been trained to classify images as either containing a blood clot or not, the NN is switched from ‘test’ mode to ‘run’ mode and used to identify blood clot events in patients.

The classification problem of finding the clot identifies the appearance of a clot. Once all the clots in an image have been identified, the next step is to determine the amount (or concentration) of the current state of clotting. One embodiment to find the concentration is to check each pixel that contains the clot. Next, find the average clot area, count all checked pixels and divide by the total pixel count. This creates a scale ranging from 0 (no clots) to 1 (filled with clots). Next, a threshold level is set that will be used to trigger an event. Assume a threshold level of 10%, so once the average clot area exceeds 0.1, an alert is posted. The alert can be used to perform a step, i.e., inject anti-coagulant to decrease the average clot area.

Once the neural network (NN) has been trained, the NN is ready for use in ECMO or for any other life threating need. Similar neural networks exist that are non-supervisory. These ML devices learn how to group events into clusters by just using data (no answers). There are many approaches to ML: Supervised learning, unsupervised learning, reinforcement learning, and feature learning, etc. There are also a number of models: NN, decision trees, support vector machine, etc.

It is understood that the above descriptions are only illustrative of the principle of the current invention. Various alterations, improvements, and modifications will occur and are intended to be suggested hereby, and are within the spirit and scope of the invention. This invention can, however, be embodied in many different forms and should not be construed as limited to the embodiments set forth herein. Rather, these embodiments are provided so that the disclosure will be thorough and complete, and will fully convey the scope of the invention to those skilled in the arts. It is understood that the various embodiments of the invention, although different, are not mutually exclusive. In accordance with these principles, those skilled in the art can devise numerous modifications without departing from the spirit and scope of the invention. The principles of clot detection as described above, can be applied to other cases, for example, in the case where blood clots can form in the blood stream even without injury. A biological specimen is defined to be a mammal. A mammal comprises humans, pigs, goats, cats, dogs, etc. The cannula is basically a tube that is inserted into the body to remove/add fluids. When inserted into veins or arteries, the fluid is blood and this blood, once removed externally, can be processed and returned to the body. The clot density is a measurement of the number of visible clots in a given area. The blood clot is also known as a thrombus. Thrombus has two components: a plug formed of platelets and red blood cells and a mesh of fibrin protein. In one embodiment, the RGB Detector components can be comprised of a light source, a light diffuser, a reflector tube, a lens, and a detector. In other embodiments, the RGB Detector can be comprised of a light source, a lens, and a detector. In some embodiments, wireless plays an important role. This inventive technique can be extended to monitor blood in the infrared spectrum range. One embodiment includes a CMOS camera and filter to detect an action in either the infrared or visible spectrum range. The action can be injecting an anti-coagulant, changing the blood flow rate, raising a flag, issuing an alarm, raising temperature, and lowering temperature. Anticoagulants may include variants of heparin, direct thrombin inhibitors, or anti-platelet drugs, Wireless communication techniques to control and interact with systems and the ability to power or charge an independent self-standing system wirelessly are well understood.

Some further definitions are provided. The detector's output presents pixel data (i.e., location, amplitude, color, etc.) where the location corresponds to the x-y position of the pixel in the array and the amplitude corresponds to the intensity of the detected light in a given frequency range. Data from all the pixels in the x-y array of the detector is collected and comprises one full scan of the detector's output. Each new full scan is an image. The image or part of the image provides the ‘pixel data output value’. The different pixels within the array capture the amplitude value of the three colors within the light that is incident at each of the different pixels for the entire array. Various algorithms can be used to find grouping of the different color intensities potentially indications a blood clot, and to assign a value to assign to the overall result which will be compared to the ‘threshold value’. A light sensitive array fabricated on a planar surface can be used to generate the ‘pixel data output value’. A reference view of blood is presented to the detector and comprises the ‘threshold value’ indicating the start of blood clotting. The ‘threshold value’ of the reference view of blood can be determined by calculating the numbers of pixels that the clot occupies then divide this number by the total number of pixels in the army. When the detector is presented a ‘threshold value’ scene, the detector's output provides the ‘reference pixel data output value’. The term ‘channeled electromagnetic radiation’ is comprised of the source light after being modified by passing through or reflected from the blood. Note that the light can also be reflected from internal sections of the blood sample. The source light illuminates the blood sample and the transmitted light and the reflected light from the blood sample comprises the ‘channeled electromagnetic radiation’. Each ‘light detection arrangement’ receives a different component or fraction of the total ‘channeled electromagnetic radiation’. As a result, different components of the ‘channeled electromagnetic radiation’ may correlate to different volumes within the blood. In one embodiment, a first ‘light detection arrangement’ receives a component of the ‘channeled electromagnetic radiation’ that the second ‘light detection arrangement’ cannot resolve, while the second ‘light detection arrangement’ receives a different component of the ‘channeled electromagnetic radiation’ that the first ‘light detection arrangement’ cannot resolve. The ‘electromagnetic radiation bandwidth’ is any range of frequencies selected within the visible spectrum range, an infrared spectrum range, or both spectrum ranges. For example, one embodiment of the ‘electromagnetic radiation bandwidth’ can be a sub-set of the visible spectrum or 405-480 THz which corresponds to the color Red.

Additionally, although the present invention is well suited for extracorporeal blood membrane oxygenation (ECMO), ECMO is but one of many possibilities of extracorporeal blood circulation systems where the present invention can be used. Furthermore, as capabilities of manufacturing the RGB detector will improve over time, sub-miniaturization techniques can be incorporated into the manufacturing of the detector until the entire unit can be reduced in size and be inserted entirely into one of the blood vessels of a patient. For example, in this case, assume 4-2 in FIG. 5C is either a vein or an artery. The RGB detector unit is attached to, coupled to, pressed against, or surgically connected to the inner walls of the vein or artery. The detector can then wirelessly communicate to the patient's mobile unit (phone) to monitor, determine and measure the blood clot formation. Power can be inductively coupled to the unit from outside the patient. The inductive coupling can transfer energy to the coils that are within the unit. The coils capture the energy.

The systems and methods disclosed herein can especially benefit from a computational machine that perform multiples and adds in parallel to significantly speed up performance. The systems and methods disclosed herein can use conventional purpose computer (but would take up to 2 orders of magnitude longer to calculate) when compared to the computational machines. These computational machines are special purpose computers that may be embedded in servers or other programmable hardware devices programmed through software, or as hardware or equipment “programmed” through hard wiring, or a combination of the two. A “computational machine” can comprise a single machine or device (a computer with multi-cores; a neural network; a gradient decent machine; machine learning device), or can comprise multiple interacting machines or processors (located at a single location or at multiple locations remote from one another). A computer-readable medium can be encoded with a computer program, so that execution of that program by one or more computers causes the one or more computers to perform one or more of the methods disclosed herein. Suitable media can include temporary or permanent storage or replaceable media, such as network-based or Internet-based or otherwise distributed storage of software modules that operate together, RAM, ROM, CD ROM, CD-R, CD-R/W, DVD ROM, DVD.+−.R, DVD.+−.R/W, hard drives, thumb drives, flash memory, optical media, magnetic media, semiconductor media, or any future storage alternatives. Such media can also be used for databases recording the information described above.

It is intended that equivalents of the disclosed exemplary embodiments and methods shall fall within the scope of this disclosure or appended claims. It is intended that the disclosed exemplary embodiments and methods, and equivalents thereof, may be modified while remaining within the scope of this disclosure or appended claims.

What is claimed is:

1. A system for a measurement of clot formation in blood comprising:
at least one light source arrangement providing an electromagnetic radiation bandwidth operating in a visible spectrum range, an infrared spectrum range, or both spectrum ranges;
a light transmission arrangement for channeling the electromagnetic radiation through, or reflected from, the blood;
one or more light detection arrangements receiving the channeled electromagnetic radiation from the light transmission arrangement to capture amplitudes over a frequency range of the channeled electromagnetic radiation; and
a computation device arrangement computing spatial, temporal, or spatial and temporal analysis on the captured amplitudes corresponding to a pixel data output value of the light detection arrangement, wherein when the pixel data output value exceeds a reference pixel data output value, an action is performed.

2. The system of claim 1, wherein the action that is performed is selected from the group consisting of injecting an anti-coagulant, changing blood flow rate, raising a flag, issuing an alarm, raising temperature, and lowering temperature.

3. The system of claim 1, wherein
two or more light detection arrangements are positioned around a volume of the blood to collect the channeled electromagnetic radiation, each capable of detecting a clotting event, the two or more of the light detection arrangements each receives a different component of the channeled electromagnetic radiation.

4. The system of claim 1, wherein
the blood being measured flows within either a cannula of an extracorporeal blood circulation system coupled to a patient, a vein of the patient, or an artery of the patient.

5. The system of claim 1, wherein
hardware for the computation device arrangement is selected from the group consisting of a field programmable gate array (FPGA), a multi-core central processing unit (MC-CPU), a graphics processing unit (GPU), and a machine learning (ML) device.

6. The system of claim 1, wherein
the light detection arrangement, further comprises:
a detector comprised of a plurality of pixels arranged in rows and columns on a planar surface; and
a lens to focus the channeled electromagnetic radiation onto the plurality of pixels, the radiation incident substantially perpendicular to the planar surface.

7. The system of claim 6, wherein
each pixel is subdivided into a red, a green, and a blue sub-pixel, and
blood clotting can be detected by using the red sub-pixel data of the pixel data output value from the light detection arrangement.

8. A system for a measurement of clot formation in blood of a patient comprising:
at least one light source arrangement providing an electromagnetic radiation bandwidth operating in a visible spectrum range, an infrared spectrum range, or both spectrum ranges;
a light transmission arrangement for channeling the electromagnetic radiation through, or reflected from, the blood of the patient; and
two or more light detection arrangements are positioned around a volume of the blood to collect the channeled electromagnetic radiation, each capable of detecting a clotting event, the two or more of the light detection arrangements each receives a different component of the channeled electromagnetic radiation from the light transmission arrangement and capturing amplitudes within a frequency range of the channeled electromagnetic radiation, wherein
when a pixel data output value of the radiation exceeds a reference pixel data output value within any of the light detection arrangements, a clotting event has been detected.

9. The system of claim 8, wherein
once the clotting event has been detected, perform an action that is selected from the group consisting of injecting an anti-coagulant, changing blood flow rate, raising a flag, issuing an alarm, raising temperature, and lowering temperature.

10. The system of claim 8, wherein
the blood being measured flows within either a cannula of an extracorporeal blood circulation system of the patient, a vein of the patient, or an artery of the patient.

11. The system of claim 8, wherein
hardware to perform the comparator operation is selected from the group consisting of a field programmable gate array (FPGA), a multi-core central processing unit (MC-CPU), a graphics processing unit (GPU), and a machine learning (ML) device.

12. The system of claim 8, wherein
the light detection arrangement, further comprises:
a detector comprised of a plurality of pixels arranged in rows and columns on a planar surface; and
a lens to focus the channeled electromagnetic radiation onto the plurality of pixels, the radiation incident substantially perpendicular to the planar surface.

13. The system of claim 8, wherein
the computation device arrangement, further comprises:
a memory to store a plurality of pixel data output values; and
a computation device arrangement computing spatial, temporal, or spatial and temporal analysis on the captured amplitudes corresponding to pixel data output value of the light detection arrangement,
a comparator to compare a reference pixel data output value and the channeled electromagnetic radiation of the blood of the patient.

14. A method for detecting and correcting a formation of a blood clot in blood comprising the steps of:
providing at least one light source arrangement that provides an electromagnetic radiation bandwidth operating in a visible spectrum range, an infrared spectrum range, or both spectrum ranges;
channeling the light through, or reflected from, the blood of the patient;
capturing amplitudes over a frequency range of the channeled electromagnetic radiation; and
computing spatial, temporal, or spatial and temporal analysis on the captured amplitudes corresponding to a pixel data output value of the light detection arrangement, wherein when the pixel data output value exceeds a reference pixel data output value, an action is performed.

15. The method of claim 14, wherein
the action that is performed is selected from the group consisting of injecting an anti-coagulant, changing blood flow rate, raising a flag, issuing an alarm, raising temperature, and lowering temperature.

16. The method of claim 14, wherein
two or more light detection arrangements are positioned around a volume of the blood to collect the channeled electromagnetic radiation, each capable of detecting a clotting event, the two or more of the light detection arrangements each receives a different component of the channeled electromagnetic radiation to detect a clotting event.

17. The method of claim 14, wherein
the blood of the patient being measured flows within either a cannula of an extracorporeal blood circulation system, a vein of the patient, or an artery of the patient.

18. The method of claim 14, wherein
hardware to computing spatial, temporal, or spatial and temporal analysis is selected from the group consisting of a field programmable gate array (FPGA), a multi-core central processing unit (MC-CPU), a graphics processing unit (GPU), and a machine learning (ML) device.

19. The method of claim 14, further comprising the steps of:
arranging a plurality of pixels in rows and columns on a planar surface; and
focusing the channeled electromagnetic radiation onto the plurality of pixels using a lens, the radiation incident substantially perpendicular to the planar surface.

20. The method of claim 19, wherein
each of the plurality of pixels comprises at least a red, a green, and a blue sub-pixel, the pixel data output of the light detection arrangement, at a minimum, only requires the response corresponding to the output of the red sub-pixel (wavelengths 625-740 nanometers) to detect blood clotting.
